(19) United States
(12) Patent Application Publication
(10) Pub. No.: US 2016/0245828 A1
(43) Pub. Date: Aug. 25, 2016

(54) Method of Identifying Biomarkers of Neurological Diseases and Diagnosis of Neurological Diseases

(71) Applicant: CRC FOR MENTAL HEALTH LTD, Victoria (AU)

(72) Inventors: Blaine Roberts, Victoria (AU); Edward A. Dratz, Bozeman, MT (US); Scott Laffoon, Bozeman, MT (US)

(73) Assignee: CRC FOR MENTAL HEALTH LTD, Victoria (AU)

(21) Appl. No.: 14/915,213

(22) PCT Filed: Aug. 27, 2014
(86) PCT No.: PCT/AU2014/000849

§ 371 Date: Feb. 26, 2016

(30) Foreign Application Priority Data
Aug. 27, 2013 (AU) 2013903257

(51) Int. Cl. G01N 33/68

(52) U.S. Cl. CPC G01N 33/6896; G01N 2800/2821; G01N 2800/2835; G01N 2800/52; G01N 2800/50; G01N 2500/04; G01N 2333/4709

(57) ABSTRACT

The present invention provides methods for identifying biomarkers of disease capable of affecting cognitive function. The biomarkers identified by the methods of the prevention may be used for predicting whether a mammal will develop a disease capable of affecting cognitive function. More specifically, the present invention relates to the identification of biomarkers predictive of neurological diseases in mammal and the use of these biomarkers in the diagnosis, differential diagnosis and/or prognosis of the neurological disease. The methods and systems provided enable an assessment and theoretical prediction of neocortical amyloid loading based on the measurement of biomarkers that will provide an indication of whether a mammal is likely to develop a neurological disease.

### CROSS-REFERENCE TO RELATED APPLICATIONS

This United States non-provisional patent application is a national stage application, which was filed under 35 U.S.C. §371 on Feb. 26, 2016 and claims priority to PCT Patent Application No. PCT/AU2014/000849, filed Aug. 27, 2014, and to AU Patent Application No. AU 2013903257, filed Aug. 27, 2013. The entirety of PCT/AU2014/000849 and AU 2013903257 are incorporated by reference herein.

### FIELD OF THE INVENTION

The present invention relates to the discovery of biomarkers. In particular the present invention provides biomarkers for the identification of a neurological disease. More particularly, the present invention relates to a method of identifying biomarkers of a neurological disease and the use of the biomarkers for the diagnosis, differential diagnosis and/or prognosis of the neurological disease.

### BACKGROUND OF THE INVENTION

Neurological disease development and progression places a significant emotional and financial burden on society.

Parkinson's disease (PD) is a common neurodegenerative disorder affecting approximately 1 in every 625 people across Western Europe. This figure rises to 4% of the population over 80. With an ageing population, the management of PD is likely to prove an increasingly important and challenging aspect of medical practice for neurologists and general physicians.

Alzheimer's disease (AD) is the most prevalent of all dementias and the third leading cause of death in Australia. The financial costs of Alzheimer's disease are estimated to be over 4 billion dollars a year in Australia while the worldwide the cost of dementia estimated to exceed $600 billion dollars.

As with other neurological diseases such as PD, clinical diagnosis of Alzheimer's disease is a difficult process as the disease progresses slowly and can take many years to manifest. Accordingly, the clinical diagnosis of Alzheimer's disease usually occurs at relatively late stages of the disease after memory and cognitive function have declined to a point that affects the patient's daily life.

The only definitive diagnosis for Alzheimer's disease is by histological examination at autopsy.

Aside from postmortem diagnosis, only two molecular diagnostic approaches are presently available. Firstly, Positron Emission Tomography (PET) is used to image markers that bind to amyloid plaques in the brain and the second is the assessment of cerebral spinal fluid (CSF) including measures of Aβ, total Tau and phosphorylated-Tau protein. However, PET and CSF are not considered viable for use in wide spread clinical practice.

For imaging AD, a series of uncharged derivatives of thioflavin T have been developed as amyloid-imaging agents and radiotracers that exhibit high affinity for amyloid deposits and high permeability across the blood-brain barrier. Extensive in vitro and in vivo studies of these amyloid-imaging agents represented by the thioflavin suggest that they specifically bind to amyloid deposits at concentrations typical of those detectable during positron emission tomography studies.

The best validated of these amyloid-imaging agents is Pittsburgh Compound-B (PiB), which is an analogue of the amyloid-binding dye Thioflavin-T. PiB-Positron Emission Tomography (PiB-PET) studies in Alzheimer's disease have shown robust cortical binding of PiB with amyloid plaque. This provides a promising early and accurate detection marker, perhaps what could be considered the gold standard. Recently other compounds have been investigated based on the similar functionality of PiB to target amyloid beta, such as AV-45 (florpiramine F-18) (otherwise known as F-18 AV-45) produced by Avid Radiopharmaceuticals Pty Ltd (Philadelphia), Floubetaben, Florbetapir, Flutematamol and NAV4694.

There have been numerous studies that have correlated the PiB radio tracer signal or output with the level of amyloid-beta and this has led to the terminology of PiB positive and PiB negative. Typically the normalisation of the PiB output, or uptake of the tracer, occurs to allow inter- and intra-subject comparisons to be made. In clinical practice normalisation for the radioactive dose and the patient's mass or volume (otherwise known as the standard uptake value (SUV)), is performed. The normalisation also incorporates standardisation with the (usually) unaffected cerebellum to provide the standard uptake value ratio (SUVR). This has led to the determination of a threshold value to differentiate those with high neocortical load (PiB positive) from those with a low load (PiB negative).

As a diagnostic test for Alzheimer's disease, the use of Pittsburgh compound B positron emission tomography (PiB-PET) imaging provides high specificity. However, due to the 11C-PiB half-life of ˜20 minutes each patient requires newly synthesised compound, restricting the use of this imaging technique to facilities equipped with comprehensive radiochemistry infrastructure, including a cyclotron. The short half-life of 11C can be partially addressed by incorporation of fluorinated compounds that are synthesised with 18F. However, the lack of a long lived-radio ligand to replace and the high cost per patient ($2000-3000/person) for PiB-PET imaging limits clinical utility of PiB-PET for the general practitioner.

Biomarkers in cerebral spinal fluid (CSF) have been found to provide confirmatory assessment of some neurological diseases for which diagnosis by imaging has been performed. Accordingly, the search for biomarkers for neurological diseases, such as Alzheimer's disease has generally focused on cerebrospinal fluid (CSF). Indeed, CSF levels of hyperphosphorylated tau and amyloid beta 1-42 (Aβ 1-42) have been shown to be predictive of conversion from MCI to Alzheimer's disease.

A drawback to using CSF is that it requires an invasive lumbar puncture to obtain a sample. In addition to being intrusive, obtaining CSF has many potential adverse outcomes for the patient. Given these limitations, it is very difficult to obtain CSF repeatedly from a large number of individuals.

A need therefore exists for an improved system capable of providing early and economically viable prognosis and/or diagnosis of neurological disease, such as Alzheimer's disease or other neurological diseases such as Parkinson's disease.

Such a system could provide assistance to clinicians in reaching an early stage prognosis and/or diagnosis prior to the portrayal of detectable clinical indicators. Moreover, with disease modifying therapies for Alzheimer's disease and Parkinson's Disease undergoing clinical trials, there is a social and economic imperative to identify biomarkers that can detect features of the disease in at-risk individuals at an early stage, so anti-Alzheimer's disease therapy or anti-Parkinson's Disease therapy can be administered at a time when the disease burden is mild and it may prevent or delay functional and irreversible cognitive loss.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE PRESENT INVENTION

There is a need for a method of identifying biomarkers for neurological diseases, particularly biomarkers that indicate the onset of the disease preferably before clinical symptoms arise. The early identification of neurological diseases could assist in delaying disease progression through early intervention.

Accordingly in an aspect of the present invention there is provided a method of identifying a biomarker of a neurological disease including

(a) isolating a first molecule with heparin binding affinity from a first sample that is positive for a neurological disease; and
(b) validating the isolated molecule as a biomarker of the neurological disease.

The present invention relates to the isolation and identification of molecules with a heparin binding affinity and the validation of these molecules as biomarkers of neurological disease. Isolating molecules with a heparin binding affinity is necessary and reduces the influence of the high abundant molecules that interfere with biomarker validation. It has now been found by the inventors that a subset of molecules with a heparin binding affinity show a high correlation with validated biomarkers of neurological diseases and further show high correlation to well established predictors of neurological diseases such as PiB/PET.

Accordingly, in performing the method of the present invention, validating the isolated molecule with heparin binding affinity as a biomarker further comprises the steps of:

(a) identifying a level of the first isolated molecule with heparin binding affinity in the first sample that is positive for a neurological disease;
(b) identifying a level of another biomarker previously defined as being characteristic for mammals diagnosed with the neurological disease present in the first sample;
(c) comparing the level of the isolated molecule identified in step (a) with the level of the other biomarker identified in step (b) to identify a statistically significant relationship between the level of the isolated molecule and the level of the other biomarker;
(d) repeating steps (a)-(c) in a second sample obtained from a control to determine whether the relationship identified in the first sample is identified in the second sample;
(e) concluding that the first isolated molecule with heparin binding affinity is a biomarker of the neurological disease if the relationship identified in the first sample is not identified in the second sample.

The presently claimed method seeks to identify a relationship between the level of an isolated molecule with heparin binding affinity and the level of another biomarker previously defined as being characteristic of a neurological disease. In performing the presently claimed method a relationship is identified by comparing the level of an isolated molecule with the level of another biomarker previously defined as being characteristic of a neurological disease. The identification of a relationship indicates that the level of the isolated molecule may also be a biomarker of the neurological disease. This can be further confirmed when compared against a control sample.

In performing the presently claimed method any relationship identified needs to be assessed to determine whether it is indicative or unique to the neurological disease by performing the same analysis in a control sample. Accordingly, the level of the isolated molecule and biomarker are identified in a control sample, the levels being compared to determine whether the relationship is identified in the control sample. If the relationship is not identified in the control sample, this indicates that the isolated molecule is likely to be a biomarker of the neurological disease.

Accordingly, in another embodiment the presently claimed method further comprises the steps of:

(a) isolating and identifying a level of a second molecule with heparin binding affinity from the first sample, the second isolated molecule being related to the first isolated molecule,
(b) generating a ratio between the levels of the first and second isolated molecules,
(c) comparing the ratio generated in step b) with the level of another biomarker previously defined as being characteristic for mammals diagnosed with the neurological disease present in the first sample to identify a statistically significant relationship between the ratio of step b) and the level of the other biomarker,
(d) repeating steps (a)-(c) in a second sample obtained from a control to determine whether the relationship identified in the first sample is identified in the second sample;
(e) concluding that the ratio is a biomarker of the neurological disease if the relationship identified in the first sample is not identified in the second sample.

Preferably, a related form of a biomarker for the determination of a neurological disease may be in one instance a protein that is present in multiple isoforms. Accordingly, it is preferred that the molecules (first and second for example) are related as isoforms.

In another aspect of the present invention there is provided a biomarker for a neurological disease, said biomarker being capable of diagnosis, differential diagnosis and prognosis of a neurological disease wherein the neurological disease is selected from the group comprising Alzheimer's disease (AD), Parkinson Disease (PD), dementia with Lewy bodies (DLB), multi-infarct dementia (MID), vascular dementia (VD), schizophrenia and/or depression. Preferably, the biomarker is capable of diagnosis, differential diagnosis and prognosis of Alzheimer's disease (AD), or Parkinson Disease (PD).

Most preferably the biomarkers for AD are selected from the group comprising antithrombin III, serum amyloid P, apoJ, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alphatrypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof. Most preferably the biomarker for AD is antithrombin III or their naturally occurring derivatives or isoforms thereof. Preferably, the isoforms are B or J of ATIII.

Most preferably the biomarker for PD is alpha-1-microglobulin (amino acids 20-203 of the alpha-1-microglobulin/bikunin precursor (AMBP)) or their naturally occurring derivatives or isoforms thereof. Preferably the isoforms of alpha-1-microglobulin are E and G.

In another aspect of the present invention, there is provided a method for diagnosis, differential diagnosis and/or prognosis of a neurological disease in a patient including:

(a) obtaining a first sample from the patient
(b) isolating and identifying a molecule with heparin binding affinity from the first sample wherein the molecule is validated as a biomarker for the neurological disease as herein described;
(c) determining whether the patient is diagnosed, differentially diagnosed and/or prognosed with the neurological disease based on the level of the molecule identified in step b).

In another aspect there is provided a method for diagnosis, differential diagnosis and/or prognosis of a neurological disease in a patient including:

(a) obtaining a sample from the patient;
(b) isolating and identifying at least two related forms of a biomarker validated according to the methods described herein from the sample;
(c) determining a level of the biomarkers from (b);
(d) generating a ratio between the levels of the two related forms of the biomarkers identified in step (b);
   (e) concluding from the ratio generated in step (d) whether the mammal is diagnosed, differentially diagnosed and/or prognosed with a neurological disease based on the ratio value compared with a reference ratio.

Accordingly, the present invention further relates to uses of biomarkers and their naturally occurring derivatives and isoforms thereof that have been identified as herein described and can be used to determine whether a mammal will possess or will be likely to develop a disease of a neurological origin or assess the mammal for cognitive deterioration.

The neurological diseases that may be considered to be of relevance to the present invention are those that would include, but are not specifically limited to, Alzheimer's disease (AD), Parkinson's Disease (PD), dementia with Lewy bodies (DLB), multi-infarct dementia (MID), vascular dementia (VD) and/or depression. A preferred disease that may be diagnosed, differentially diagnosed and/or prognosed through the use of the methods of the present invention is AD or PD.

In a further preferred embodiment of the present invention, the method further includes the steps of:

(a) obtaining a first sample from a patient;
(b) isolating and identifying a level of a first and second biomarker with heparin binding affinity from the first sample, wherein the first and the second biomarkers are related and wherein the first and second biomarkers are validated as a biomarker for the neurological disease as herein described,
(c) generating a ratio between the levels of the first and second biomarkers to provide a generated ratio,
(d) repeating steps (b)-(c) in a second sample obtained from a control to provide a reference ratio,
   (e) comparing the generated ratio identified in the first sample with the reference ratio identified in the second sample;
(f) concluding a neurological disease status based on a difference between the generated ratio and the reference ratio.

In the methods of the present invention, at least two biomarkers associated with one or more neurological diseases including antithrombin III, serum amyloid P, apo J (clusterin), alpha-1-microglobulin or their naturally occurring derivatives or isoforms thereof are quantified in the generation of a ratio to indicate a neurological disease state of a mammal.

In a further aspect of the present invention there is provided a method for monitoring the progression of a neurological disease in a mammal; methods for stratifying or identifying a mammal at risk of developing a neurological disease; and methods for screening for agents that interact with and/or modulate the expression or activity of a biomarker associated with a neurological disease.

In a further aspect, the present invention provides a kit that can be used for the diagnosis and/or prognosis in a mammal of one or more neurological diseases or for identifying a mammal at risk of developing one or more neurological diseases.

Other aspects of the present invention will become apparent to those ordinarily skilled in the art upon review of the following description of specific embodiments of the invention.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

### DESCRIPTION OF THE TABLES AND FIGURES

For a further understanding of the aspects and advantages of the present invention, reference should be made to the following detailed description, taken in conjunction with the accompanying drawings.

FIG. 1 shows the 2D gel analysis of protein analytes with the tentative nomenclature used throughout this application. Antithrombin III is abbreviated as “AT” to refer to all variants in highlighted Antithrombin III series (A-AH), that includes Antithrombin III and possible variants from other proteins. “ApoJ” refers to the associated, highlighted and unidentified protein variants A-G. “SAP” refers to the associated, highlighted serum amyloid protein variants A-K.

FIG. 2 shows 2-DGE studies. Clinical classification—Comparison of the mean ratio between antithrombin III isoforms A and J demonstrates a highly significant difference between AD and controls. The ratio of the most basic isoform A and isoform J of antithrombin III is significantly elevated in patients clinically diagnosed with mild cognitive impairment and Alzheimer's disease compared to cognitively normal individuals. (Anova Tukey post-hoc, Mean+/−stdev).

FIG. 3 shows Classification by PiB-SUVR—ATIII A/J ratio Plasma (t-test, Mean+/−stdev). Correlation of antithrombin III isoforms and standard uptake value ratio (SUVR) for Pittsburgh compound-B (PiB) positron emission tomography (PET) in the brain of 73 subjects involved in the AIBL study (A).

FIG. 4—Representative gel images from six RP sub-fractions after MARS14 depletion. The arrows indicate the protein changes in AD pools.

FIG. 5—False-color image overlays of unaligned F2 multiplex gels. Three chains of Hpt are shown in ovals in the upper right image.

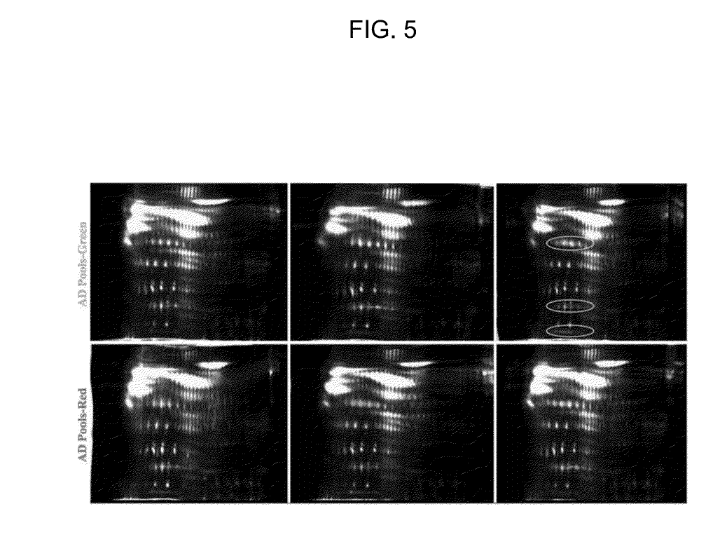

FIG. 6—Detail from multiplexed gel images representative of A Low ApoE 4 containing pools and B. High ApoEa4 containing pools. ±1ACT isoforms correlated with the 34 kDa ApoE a4 proxy spot, shown in lower right-hand corners of these images. Regression analysis correlations: a—p=0.012, R2=0.45; b—p=0.002, R2=0.61; c—p=0.003, R2=0.56; d—p=0.002, R2=0.61; e—p=0.007, R2=0.51; f—p=0.003, R2=0.58. None of the ±1ACT spots significantly discriminated AD from HC in the pooled experiment. The ±1AT spot that significantly discriminated AD from control pools (3.3 fold, p<0.02) is shown in the lower image.

FIG. 7—Intact and cleaved VDBP with sex specific changes shown in tables on the right. A. Intact (top spot train) and cleaved VDBP (A,B,C). The intact VDBP spots were saturated and masked from the Progenesis analysis. B. Cleaved VDBP (A-M). C. Cleaved VDBP (A-E).

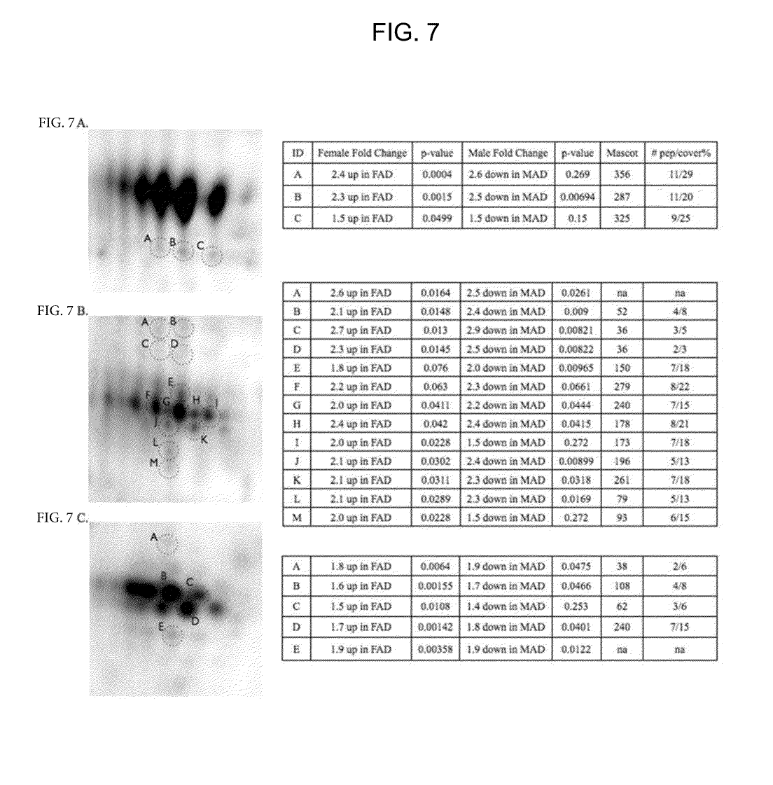

FIG. 8—AD Biomarkers (ATIII, ApoJ and SAP) are not elevated in PD plasma.

FIG. 9—ApoJ correlates with Aβ.

FIG. 10—Levels of Alpha-1-microglobulin (AMBP) are elevated in Parkinson's disease plasma. The level of AMBP between control (n=37) samples and PD (n=44) samples (top-left mean, STDEV) is significantly elevated in PD plasma (p<0.0001). The dashed line indicates the cut-off value to above which individuals would be considered to have PD. The ROC analysis of AMBP levels is shown in the top right. The bottom figure is the correlation of the AMBP levels with clinical unified Parkinson's disease rating scale (UPDRS). Statistical analysis was conducted using Prism v5.0f. Statistical test used was t-test p-value greater than 0.05 was considered significant. The intensity for isoform E is shown in this figure. Similar results are obtained for isoform G for AMBP.

FIGS. 11—2D spot map for AMBP.

FIG. 12—Comparison of ratio 193/166 (G/E) between PD and controls. The dashed line represents 80% specificity of the test and individuals at the cutoff value have a 5.0 odds ratio. (n=31 controls n=51 PD).

TABLE 1 shows the ROC analysis summary for AD biomarkers.

TABLE 2 shows proteins that had at least one isoform meeting the inclusion criteria for change between AD and control controls and CRP isoforms. Haptoglobin was identified from a preparative gel of unreduced complex. NS—not significant.

TABLE 3 shows biomarkers for brain amyloid discovered using mass spectrometry.

### DETAILED DESCRIPTION OF THE INVENTION

The present invention provides methods of identifying biomarkers for diagnosis, differential diagnosis and/or prognosis of neurological diseases that are predictive of cognitive deterioration, by isolating molecules with a heparin binding affinity from a sample obtained from a mammal. These biomarkers are related to and correlate with amyloid loading. The biomarkers identified in the present invention can be used to diagnose amyloid in the brain or to detect changes in amyloid levels in the brain. Once identified, the marker may be used in high throughput diagnostic or prognostic tests for amyloid in the brain.

Accordingly in an aspect of the present invention there is provided a method of identifying a biomarker of a neurological disease including

(a) isolating a first molecule with heparin binding affinity from a first sample that is positive for a neurological disease; and
(b) validating the isolated molecule as a biomarker of the neurological disease.

The present invention relates to the isolation and identification of molecules with a heparin binding affinity and the validation of these molecules as biomarkers of neurological disease. Isolating molecules with a heparin binding affinity is necessary and reduces the influence of the high abundant molecules that interfere with biomarker validation. It has now been found by the inventors that a subset of molecules with a heparin binding affinity show a high correlation with validated biomarkers of neurological diseases and further show high correlation to well established predictors of neurological diseases such as PiB/PET.

As would be understood by one of skill in the art, a biomarker is regarded as an indicator of a biological state of a particular mammal, or a patient, or a subject or an individual. It is considered that terms such as ‘mammal’, ‘patient’, ‘subject’ or ‘individual’ are also terms that can, in context, be used interchangeably in the present invention. It is further considered that the terms ‘individual’ and ‘subject’ can be used interchangeably to refer to the same test subject being examined or analysed for the presence of biomarkers and evaluated in determining the status of a neurological disease.

Moreover, a biomarker need not be an individual molecule. While a biomarker may be a single molecule it may also be a plurality of molecules. When considering a biomarker as a plurality of molecules, the biomarker may relate to a representation of a relationship between the molecules. For example, the relationship may be a ratio. Furthermore, the plurality of molecules may represent a molecular signature that is indicative of a neurological disease. More particularly, the signature may be defined by the expression level of a plurality of proteins or protein isoforms.

A biomarker can be further regarded as being a particular characteristic that could be objectively measured and evaluated as an indicator of, for instance, a normal biological process, a pathogenic process, or a pharmacologic response to a therapeutic intervention in a mammal. Often, where the use of a single biomarker is not capable of completely determining whether a mammal possess or is absent a neurological disease, the presence and/or absence of two or more biomarkers may be required for the appropriate derivation of the biological state for the mammal.

Biomarkers, alone or in combination, can also provide measures of relative risk that a mammal belongs to one phenotypic status or another. Therefore, biomarkers are conventionally useful for indicating the likelihood that a mammal will develop a disease (prognostic), possess a disease (diagnostic) or ascertain the therapeutic effectiveness of a drug (theranostic) and drug toxicity.

A biomarker would also be considered to include, but is not necessarily be limited to, proteins, polypeptides, polynucleotides and/or metabolites present in a biological sample whose level (e.g., concentration, expression and/or activity) in a sample from a mammal or a control population is indicative of a biological state, for example diagnostic for a neurological disease. Further, biomarkers contemplated within the methods of the present invention, can also include, but are not necessarily limited to, immunoglobulins, peptides, mRNA, DNA, small non-coding RNA, miRNA, digested protein fragments, enzymes, lipids, metabolites, carbohydrates, glycosylated polypeptides, and metals.

The presently claimed method may identify biomarkers in neurological disorders associated with increased neocortical amyloid. In a preferred embodiment of the invention the neurological diseases that may be considered to be of relevance to the present invention are those that would include, but are not specifically limited to, Alzheimer's disease (AD), Parkinson Disease (PD), dementia with Lewy bodies (DLB), multi-infarct dementia (MID), vascular dementia (VD), schizophrenia and/or depression. Diagnosis and prognosis of neurological diseases such as AD and PD through the use of the methods of the present invention are particularly desired. It is also desired that the biomarkers identified and/or isolated reflect the PiB load in the brain.

In performing the presently claimed method, molecules are isolated based on their heparin binding affinity, their affinity for heparin or their association with molecules that are attracted to heparin. In the context of the present invention, terms such as obtaining, extracting, purifying and removed are synonymous with the term isolating. Moreover, it is considered that terms such as ‘heparin binding affinity’ or ‘affinity for heparin’ are terms that can be used interchangeably in the present invention. In the context of the present invention, affinity is defined as an attraction or force between molecules that causes them to associate or bind. Accordingly, a molecule isolated by the presently claimed method would have such an attraction to heparin. Hence, molecules that have heparin binding affinity will include molecules that directly associate with heparin or are associated, bound or complexed to other molecules that are attracted to heparin.

Applicants have identified that molecules having an affinity for heparin can be indicative of neurological diseases such as but not limited to Alzheimer's disease (AD), Parkinson Disease (PD), dementia with Lewy bodies (DLB), multi-infarct dementia (MID), vascular dementia (VD), schizophrenia and/or depression. More preferably the molecules can be indicative of AD and/or PD. Hence these molecules can present as biomarkers for these neurological diseases.

The description that follows generally relates to AD and PD. However, the methods described herein are equally applicable to other neurological diseases and the identification of biomarkers for those neurological diseases.

The heparin binding affinity of a molecule is used to select out or isolate specific molecules from a mixture or sample of non-heparin-binding molecules or molecules without an affinity for heparin. Accordingly, in the context of the present invention, molecules need only have sufficient heparin binding affinity to be isolated from a sample or mixture of molecules without an affinity for heparin.

In isolating molecules with a heparin binding affinity the molecules may non-covalently or covalently bind to heparin. As an example, heparin may be immobilised to select or isolate molecules from a sample based on their heparin binding affinity leaving molecules without an affinity for heparin in the sample. In other examples, molecules with a heparin binding affinity may be isolated by using antibodies, peptide arrays, molecular imprinting, or a chemical affinity matrix.

As would be appreciated by one of skill in the art, the format of immobilized heparin can vary widely. For example, heparin may be immobilised on a coated surface or included in a chromatography resin.

A molecule may be isolated by its association or binding with immobilised heparin or may associated, bound or complexed to another molecule that is attracted to heparin. Alternatively, immobilised heparin may act as a high-capacity cation exchanger. This use takes advantage of heparin's high number of anionic sulfate groups. These groups will capture molecules or proteins with an overall positive charge. Methods and apparatus for isolating molecules based on their affinity for heparin would be known to the skilled addressee. Preferably, an apparatus or assay which provides free heparin for binding molecules with an affinity for heparin is used in the presently claimed method. More preferably, a heparin-sepharose purification column is used to isolate molecules with a heparin binding affinity.

Molecules may bind to heparin and then be selectively dissociated from heparin with the use of various buffering conditions such as varied pH or salt concentration or by use of a gradient such as a salt or pH gradient.

As one of skill in the art would appreciate, isolated proteins can be selectively isolated from a sample using a heparin-sepharose purification column by varying the columns' pH. Accordingly, in another aspect, the heparin-sepharose column is eluted at least about pH 3, at least about pH 4, at least about pH 5, at least about pH 6, at least about pH 7, at least about pH 8, at least about pH 9, at least about pH 10. More preferably the heparin sepharose column is eluted at pH 6 to pH 8, more preferably at pH 7 or pH 8.

Alternatively, heparin may be dissolved in a sample, selectively binding molecules with a heparin binding affinity in the sample. Subsequent purification of the heparin bound molecules could then be used to isolate these molecules from the sample. Isolated molecules may then be selectively dissociated from heparin before identifying their level.

As would be understood by one of skill in the art, affinities can be influenced by non-covalent intermolecular interactions between at least two molecules. Accordingly, a dissociation constant may be used to describe the affinity between a molecule and heparin (i.e. how tightly a molecule associates or binds to heparin). Hence molecules with varying degrees of heparin binding may be isolated as potential biomarkers.

Alternatively, in performing the claimed invention, a molecule may be isolated based on it encoding a sequence of a known heparin binding region such as a heparin binding domain. For example, in such an alternative, PCR primers directed to the heparin binding domain may be designed to amplify molecules containing or encoding such regions. These molecules may be purified and analysed to determine their level of expression.

As one of skill in the art would appreciate, heparin is a mixture of linear anionic polysaccharides having 2-O-sulfo-α-L-iduronic acid, 2-deoxy-2-sulfamino-6-O-sulfo-α-D-glucose, β-D-glucuronic acid, 2-acetamido-2-deoxy-α-D-glucose, and α-L iduronic acid as major saccharide units. The presence and frequency of these saccharide units vary with the tissue source from which heparin is extracted. However, performance of the present invention is not intended to be limited to a specific isoform, subtype or species of heparin. Accordingly, heparin used in the context of the present invention may be isolated and purified from various cell or tissue samples from various species. Alternatively, heparin may be obtained from cultured cells. Alternatively, the heparin may be semi-synthetic or synthetic.

In another preferred embodiment, the first sample may be pretreated to remove or reduce the influence of high abundant proteins that interfere with proteomic analysis prior to isolating molecules with heparin binding affinity. As an example, the samples may be treated with the multiple affinity removal system-14 (MARS), which removes at least the most abundant proteins from the sample. This then provides an improved enrichment process which utilizes the heparin binding affinity of potential biomarkers.

It is contemplated that the sample used in the present invention be a biological sample. In the context of the present invention, the sample can be obtained from a mammal. The sample may include a variety of biological materials selected from but not limited to the group consisting of blood (including whole blood), blood plasma, blood serum, hemolysate, lymph, synovial fluid, spinal fluid, urine, cerebrospinal fluid, semen, stool, sputum, mucus, amniotic fluid, lacrimal fluid, cyst fluid, sweat gland secretion, bile, milk, tears or saliva. Preferably, the biological sample is blood (including whole blood), blood plasma, or blood serum.

Moreover, the skilled addressee would be aware that the presently claimed methods could be used in any obtained biological material containing DNA, RNA and/or protein.

More preferably, the isolated molecule is selected from the group consisting of immunoglobulins, peptides, mRNA, small non-coding RNA, miRNA, DNA, digested protein fragments, enzymes, metabolites, carbohydrates, glycosylated polypeptides, or metals.

The mammal examined through the methods of the present invention may be a human mammal or a non-human mammal. A non-human mammal may be, but is not necessarily considered limited to, a cow, a pig, a sheep, a goat, a horse, a monkey, a rabbit, a hare, a dog, a cat, a mouse or a rat. In one embodiment, the mammal is a primate. In preferred embodiment the mammal is a human, more preferably the mammal is a human adult.

The method of the present invention can also be used in animal models representative for a human disease, for example, for use in in-vivo models of biomarker identification. In such an embodiment, the animal in the animal model is a mouse, a rat, a monkey, a rabbit, an amphibian, a fish, a worm, or a fly.

In performing the presently claimed method of identifying biomarkers of neurological diseases the sample obtained from a mammal is positive or potentially positive for a neurological disease. Preferably, clinical and/or molecular diagnosis can be used to confirm that the mammal from which the sample was obtained is positive for a neurological disease. This includes mammals that are cognitively normal but show changed levels of a marker indicative of a neurological disease such as amyloid loading in the brain (preferably determined by PET imaging). These mammals are potentially positive for a neurological disease and are included in the scope of the present invention.

It would be understood by one skilled in the art that clinical determinations used to determine whether the mammal is positive or potentially positive for a neurological disease would be considered to relate to assessments that include, but are not necessarily limited to, memory and/or psychological tests, assessment of language impairment and/or other focal cognitive deficits (such as apraxia, acalculia and left-right disorientation), assessment of impaired judgment and general problem-solving difficulties, assessment of personality changes ranging from progressive passivity to marked agitation.

Moreover, a positive diagnosis of a disease state of a mammal can be validated or confirmed if warranted, such as determining the amyloid load or amyloid level to confirm the presence of high neocortical amyloid. The terms amyloid load or amyloid level, often used interchangeably, or presence of amyloid and amyloid fragments, refers to the concentration or level of cerebral amyloid beta (Aβ or amyloid-β) deposited in the brain, amyloid-beta peptide being the major constituent of (senile) plaques.

A mammal can also be confirmed as being positive for a neurological disease using imaging techniques including, PET and MRI, or with the assistance of diagnostic tools such as PiB when used with PET (otherwise referred to as PiB-PET). Preferably, the mammal positive for a neurological disease is PiB positive. More preferably, the mammal has a standard uptake value ratio (SUVR) which corresponds with high neocortical amyloid load (PiB positive). For instance, current practice regards a SUVR can reflect 1.5 as a high level in the brain and below 1.5 may reflect low levels of neocortical amyloid load in the brain. A skilled person would be able to determine what is considered a high or low level of neocortical amyloid load. As would be appreciated by one of skill in the art, a mammal can also be confirmed as being positive for a neurological disease by measuring amyloid beta and tau from the CSF.

For the purposes of identifying a biomarker of a neurological disease, samples may be obtained from a library of samples which have been positively identified as being obtained from patients diagnosed with a neurological disease such as AD and PD and the amyloid levels may also have been determined. Suitable libraries may include The Australian Imaging, Biomarker and Lifestyle (AIBL) Flagship study of Aging or The Alzheimer's Disease Neuroimaging Initiative (ADNI).

To date, AIBL has involved evaluating approximately 1,112 volunteers across four dimensions including neuroimaging, biomarkers, psychometrics, and lifestyle factors. The AIBL study is a longitudinal study with blood draws at 18-month intervals over a period of eight years. It is the largest study in the world involving positron emission tomography (PET) scans using the amyloid-imaging agent, Pittsburgh compound-B (PiB). One advantage that the AIBL has over other similar studies is a standardized procedure for the collection and storage (liquid N2) of the blood samples. This is a significant advantage in comparison to other studies that have varied collection and storage protocols or store samples at −20C. The AIBL study presents a rich resource of well-characterized blood samples from AD, mild-cognitively impaired (MCI), and unimpaired age-matched control subjects that offer an excellent resource for the discovery of biomarkers that can be used for diagnosis of AD and PD.

ADNI is a study of AD designed to validate the use of biomarkers from blood, cerebrospinal fluid, magnetic resonance imaging (MRI) and positron emission tomography (PET) imaging. ADNI, like AIBL, has collected longitudinal blood samples and a battery of neuropsychometric data on participants.

As would be understood by the skilled addressee, an isolated molecule is validated as a biomarker when its level, alone or in combination is considered statistically relevant or if its relationship with other previously characterised biomarkers distinguishes phenotypic statuses. The usefulness of an identified biomarker for determining a disease status is considered statistically significant when the probability that the particular molecule has been identified as a biomarker by chance is less than a predetermined value. The method of calculating such probability will depend on the exact method utilised to compare the levels of the biomarkers.

There are a number of statistical tests for identifying biomarkers that vary significantly, including the conventional t-test. However, it may be generally more convenient, appropriate and/or accurate to use a more sophisticated technique, such as SAM or Prediction Analysis of Microarray (PAM) (http://www-statstanford.edu/.about.tibs/PAM/index.html), or Random Forests. Common tests to assess for such statistical significance include, among others, t-test, ANOVA, Kruskal-Wallis, Wilcoxon, Mann-Whitney and odds ratio.

In performing the method of the present invention, in one embodiment, validating the isolated molecule with heparin binding affinity may involve comparing a statistically significant difference in a level of an isolated molecule with heparin binding affinity between a sample positive or potentially positive for a neurological disease with a control.

Accordingly, in another embodiment in performing the method of the present invention, validating the isolated molecule with heparin binding affinity as a biomarker further comprises the steps of:

(a) identifying a level of the first isolated molecule with heparin binding affinity in the first sample that is positive or potentially positive for a neurological disease; (b) identifying a level of another biomarker previously defined as being characteristic for mammals diagnosed with the neurological disease present in the first sample;
(c) comparing the level of the isolated molecule identified in step (a) with the level of the other biomarker identified in step (b) to identify a statistically significant relationship between the level of the isolated molecule and the level of the other biomarker;
(d) repeating steps (a)-(c) in a second sample obtained from a control to determine whether the relationship identified in the first sample is identified in the second sample;
(e) concluding that the first isolated molecule with heparin binding affinity is a biomarker of the neurological disease if the relationship identified in the first sample is not identified in the second sample.

In performing the presently claimed method the level of the isolated molecule and biomarker must be identified. As would be appreciated by one of skill in the art, the level (e.g., concentration, expression and/or activity) of an isolated molecule or the previously identified biomarker can be qualified or quantified. Preferably, the level of the isolated molecule or biomarker is quantified as a level of DNA, RNA, lipid, carbohydrate, metal or protein expression. In this preferred embodiment, the present invention seeks to validate isolated molecules as biomarkers based on their respective expression level having a statistically significant relationship with the level of a biomarker previously defined as being characteristic for mammals diagnosed with the neurological disease.

It will be apparent that numerous qualitative and quantitative techniques can be used to identify the level of the isolated molecules and biomarkers. These techniques may include 2D DGE, mass spectrometry (MS) such as multiple reaction monitoring mass spectrometry (MRM-MS), Real Time (RT)-PCR, nucleic acid array; ELISA, functional assay, by enzyme assay, by various immunological methods, or by biochemical methods such as capillary electrophoresis, high performance liquid chromatography (HPLC), thin layer chromatography (TLC), hyper-diffusion chromatography, two-dimensional liquid phase electrophoresis (2-D-LPE) or by their migration pattern in gel electrophoreses. Sodium dodecyl sulfate-polyacrylamide gel electrophoresis (SDS-PAGE) is a widely used approach for separating proteins from complex mixtures.

However, it will be apparent to the skilled addressee that the appropriate technique used to identify the level of the isolated molecules and biomarkers will depend on the characteristics of the molecule. For example, if the isolated molecule is a protein, 2D DGE or Mass spectrometry may be used to quantify the level of the isolated molecule.

Preferably the quantification of the levels of a biomarker can be performed in one- or two-dimensional (2-D) configuration. For less complicated protein preparation, one-dimensional SDS-PAGE is preferred over 2-D gels, because it is simpler. In a preferred embodiment, 2-D gel electrophoresis is utilised which incorporates isoelectric focusing (IEF) in the first dimension and SDS-PAGE in the second dimension, leading to a separation of the biomarkers by charge and size.

The determination of the level of a biomarker may also be made by, for example, following characterisation of the biomarker based on their isoelectric focusing point (pi) and their molecular weight (MW), such as on 2-D gel electrophoresis if the biomarker were a polypeptide. In this example, the amount of a biomarker present in a sample could be determined through visual analysis, such as by measuring the intensity of a polypeptide spot on a 2-D gel.

In one example, a quantitative technique such as RT-PCR can conceivably be used by one of skill in the art to assess the quantity of a biomarker if the biomarker were a polynucleotide biomarker. In another example if the particular biomarker were a polypeptide or protein, the level of the biomarker could be determined through ELISA techniques utilising a secondary detection reagent such as a tagged antibody specific for the polypeptide biomarker.

In a non-limiting example where the biomarker is protein, the level of protein or protein isoform can also be detected by an immunoassay. An immunoassay would be regarded by one skilled in the art as an assay that uses an antibody to specifically bind to the antigen (i.e. the protein or protein isoform). The immunoassay is thus characterised by detection of specific binding of the proteins or protein isoforms to antibodies. Immunoassays for detecting proteins or protein isoforms may be either competitive or non-competitive. Non-competitive immunoassays are assays in which the amount of captured analyte (i.e. the protein or protein isoform) is directly measured. In competitive assays, the amount of analyte (i.e. the protein or protein isoform) present in the sample is measured indirectly by measuring the amount of an added (exogenous) analyte displaced (or competed away) from a capture agent (i.e. the antibody) by the analyte (i.e. the protein or protein isoform) present in the sample.

In one example of a competition assay, a known amount of the (exogenous) protein or protein isoform is added to the sample and the sample is then contacted with the antibody. The amount of added (exogenous) protein or protein isoform bound to the antibody is inversely proportional to the concentration of the protein or protein isoform in the sample before the exogenous protein or protein isoform is added. In another assay, for example, the antibodies can be bound directly to a solid substrate where they are immobilized. These immobilised antibodies then capture the protein or protein isoform of interest present in the test sample. Other immunological methods include but are not limited to fluid or gel precipitation reactions, immunodiffusion (single or double), agglutination assays, immunoelectrophoresis, radioimmunoassays (RIA), enzyme-linked immunosorbent assays (ELISA), Western blots, liposome immunoassays, complement-fixation assays, immunoradiometric assays, fluorescent immunoassays, protein A immunoassays or immunoPCR.

Alternatively, it is contemplated that secondary measurement processes could be utilised for the determination of the biomarker in a given sample. For example, if a biomarker is a protein with enzymatic properties, a measurement of the enzymatic activity could be possibly utilised in determining the level of the biomarker. Similarly, if the biomarker were a polypeptide, it is considered that the level of the biomarker could be made through a measure of mRNA coding for the polypeptide. Qualitative data may also be derived or obtained from primary measurements.

Alternatively, if the isolated molecule is a miRNA RT-PCR may be used. In a preferred example, the isolated molecule is a protein and its expression is measured using 2D DGE. In this preferred embodiment the molecule is labelled with an amine reactive or thiol reactive zwiterionic fluorescent dye Zdye prior to quantifying the level of expression of the molecule.

Biomarkers present in a sample can be quantified to obtain a level by using individual multicolour, differential in-gel electrophoresis (DGE). DGE detection on 2-D gels has the advantage that it avoids the problem of gel-gel variability through the inclusion of an internal standard on each gel and can be carried out with many fewer gels. Additionally, there are few techniques that can resolve as many proteins from a single sample as conventional 2-D gels.

In quantitating the level of the isolated molecule the sample, either prior to or after isolation of a molecule with heparin binding affinity, may be treated to improve precision for quantitative assays such as for 2D gels and mass spectrometry. The enriched proteins from a heparin sepharose column may be reduced and alkylated using reducing and alkylating agents such as but not limited to tris(2-carboxyethyl) phosphine (TCEP) and 4-vinylpyridine followed by enzymatic digestion with trypsin (preferably overnight at about 37° C.). Peptides for multiple reaction monitoring may be determined using MS data, the resource Skyline and peptide transitions for quantitative measurement of peptides with heparin binding affinity such as, but not limited to apoE, apoJ, antithrombin III, serum amyloid P, fibrinogen and Aβ. In addition to these proteins, others such as, actin, gelsolin and apoE can be measured.

Skyline is a software resource that aids in the rapid selection of peptides suitable for development of quantitative MS. The digested proteins are serially diluted and detection limit, ionisation efficiency, reproducibility and chromatographic behaviour are determined using nano-LC-MRM (Q-trap 6500, ABSciex). For the quantitative assay peptides may be synthesised with isotopically labelled lysine or arginine amino acids. The isotopically labelled peptides (heavy peptides) may be labelled with 13C and 15N to produce a mass shift of 8-10 Da. The mass spectrometer may resolve, the otherwise identical peptide, based on the mass difference. The heavy peptides serve as a true internal standard as they are chemically identical to the peptides in the sample; this is one of the major advantages of MRM-MS. Amino acid analysis is used to determine peptide concentrations.

Without being limited by theory, the present invention is based on the finding that levels of molecules with heparin binding affinity are altered in a sample obtained from a mammal determined as having a neurological disease when compared to the levels of the same molecules in a sample obtained from a mammal that is determined not to possess the same neurological disease. Moreover, these alterations correlate with the level of biomarkers previously defined as being characteristic for mammals diagnosed with the neurological disease.

Accordingly, in performing the claimed methods, the level of an isolated molecule may be compared with known biomarkers which correlate with the presence of high neocortical amyloid. Preferably, the comparison is made with a level of a radiotracer specifically recognising the presence of the amyloid beta in brain. Such a radiotracer may be Pitsburg compound B (PiB) or florpiramine F-18. More preferably, the comparison is made with a PiB-PET level which is characteristic of the neurological disease.

The biomarker being characteristic for mammals diagnosed with the neurological disease may also be a previously determined ratio (reference ratio) of biomarkers from samples possessive of the neurological disease state. For example, the comparison can be made with a SUVR>1.5 or any other determined value that reflects a high or low amyloid loading as determined by the skilled addressee. Above this amount, the amyloid loading may be considered to be high and low, it may be considered to be low. However, this application is not limited to this value.

Alternatively, the level of an isolated molecule may be compared with the level of any of one or more additional known biomarkers for neurological diseases, including but not limited to amyloid β peptides, tau, phospho-tau, synuclein, Rab3a, and neural thread protein. Moreover, the comparison may be made against clinical biomarkers values such as Clinical Dementia Rating (CDR) or Body Mass Index from which the set of biological samples was obtained.

As will be understood in the practice of the methods of the present invention, the comparison need not be limited to a single biomarker characteristic of the neurological disease. Including further biomarkers in the comparison may reduce the risk of false positive biomarker identification. Accordingly, it is contemplated in a preferred feature of the claimed methods that additional biomarkers characteristic of the neurological disease will also be compared to the level of the isolated molecule to identify a relationship.

The presently claimed method seeks to identify a relationship between the level of an isolated molecule with heparin binding affinity and the level of another biomarker previously defined as being characteristic of a neurological disease. In performing the presently claimed method a relationship is identified by comparing the level of an isolated molecule with the level of another biomarker previously defined as being characteristic of a neurological disease. The identification of a relationship indicates that the level of the isolated molecule may also be a biomarker of the neurological disease. This can be further confirmed when compared against a control sample.

The relationship may be appreciated from a side by side comparison. For example, the level of the isolated molecule may change in a similar or related magnitude or direction with respect to the known biomarker. Preferably, the relationship is a correlation. While the skilled addressee would be aware of particular means and methods for identifying correlations between data sets, examples of correlation methods include Pearson's correlation and Rank correlation coefficients such as Spearman and Kendall tau. Moreover, the correlation need not be linear or define a linear relationship. The relationship may also be non-linear and may be apparent when analysing at a data set graphically.

More particularly, the method of the present invention seeks to validate isolated molecules as biomarkers based on a relationship or correlation with the increased presence of amyloid and/or amyloid fragments, such as beta amyloid, in the neocortex of a mammal.

More particularly, the present invention validates isolated molecules as biomarkers based on their relationship or correlation with measurements obtained from PiB-PET studies or AV-45 measurements. PiB-PET studies may also define increased presence of amyloid and/or amyloid fragments in terms of high-PiB relative to low-PiB correlating with reduced presence of amyloid and/or amyloid fragments. Preferably, in performing the presently claimed method and validating the isolated molecules as biomarkers, the level of the isolated molecule correlates with a high-PiB measurement.

In performing the presently claimed method any relationship identified needs to be assessed to determine whether it is indicative or unique to the neurological disease by performing the same analysis in a control sample. Accordingly, the level of the isolated molecule and biomarker are identified in a control sample, the levels being compared to determine whether the relationship is identified in the control sample. If the relationship is not identified in the control sample, this indicates that the isolated molecule is likely to be a biomarker of the neurological disease.

To conclude whether an isolated molecule is a biomarker of the neurological disease the relationship identified in the sample positive for the neurological disease will not be identified or present in the control sample. Accordingly, the relationship is indicative of the sample obtained from a mammal positive for the neurological disease and not indicative of the control sample.

Broadly, in performing the presently claimed method, the results obtained from an experimental sample, are compared against a control sample. In the context of the present invention, the experimental sample represents a sample obtained from a mammal positive for a neurological disease.

The control sample may be a biological sample either positive or negative for the neurological disease. However, as one of skill in the art would appreciate, the control sample is dictated by the experimental sample in that it must provide the necessary comparison for validating an isolated molecule as a biomarker of neurological disease. For example, if the experimental sample is positive for the neurological disease the control sample would ideally be negative for the neurological disease. In being negative for the neurological disease, the control sample may be from a healthy mammal that has no symptoms of neurological disease. Alternatively, the control sample may be from a mammal that has an alternative neurological disease. For instance, when validating an AT biomarker, the control sample may be a PD sample.

Furthermore, the experimental and control samples may consist of a plurality of samples to form experimental and control groups. Accordingly, validating the isolated molecule as a biomarker the level of the first isolated molecule and the other biomarker may be identified in a group of samples comprising the experimental group and another group of samples comprising the control group. The sample size for the experimental and control group need not be equal.

Moreover, the control group need not comprise the same samples so long as the samples are distinguished from the experimental group. For example the control group may consist of samples from healthy mammals without neurological disease and mammals with an alternative neurological disease to the control group. For example, the experimental group can contain samples from mammals with PD and the control group can contain samples from healthy mammals without neurological disease and samples from mammals with AD.

The present inventors have found that the comparison of the levels of additional isolated molecules with a heparin binding affinity in a sample obtained from a mammal positive for a neurological disease to provide a ratio may provide biomarkers of the neurological disease. These biomarkers may have increased specificity and sensitivity in diagnosing the neurological disease when compared with the use of the levels of the molecules individually.

Accordingly, in another embodiment the presently claimed method further comprises the steps of:

(a) isolating and identifying a level of a second molecule with heparin binding affinity from the first sample, the second isolated molecule being related to the first isolated molecule,
(b) generating a ratio between the levels of the first and second isolated molecules,
(c) comparing the ratio generated in step b) with the level of another biomarker previously defined as being characteristic for mammals diagnosed with the neurological disease present in the first sample to identify a statistically significant relationship between the ratio of step b) and the level of the other biomarker, (d) repeating steps (a)-(c) in a second sample obtained from a control to determine whether the relationship identified in the first sample is identified in the second sample;
(e) concluding that the ratio is a biomarker of the neurological disease if the relationship identified in the first sample is not identified in the second sample.

As considered in the present invention, the validation of a biomarker ratio of neurological disease comprises measuring the level of at least one isolated molecule, correlating that level to the level of at least one other related isolated molecule, and determining the ensuing mathematical relationship.

The ratio is then compared with the level of a biomarker previously defined as being characteristic of the neurological disease to identify a relationship. This relationship is subsequently assessed in a control sample to conclude whether the ratio is a biomarker of neurological disease.

In the presently claimed methods, related forms of the molecules such as the second molecule or the second isolated molecule are those that have a degree of similarity, can be derived from the same origin molecule, and/or can be grouped together due to a shared property or attribute to another molecule such as the first molecule or the first isolated molecule. For example, in the context of a polypeptide, related biomarkers indicative of a disease state can include polypeptides which are based or derived from the same parent molecule (for example, encoded from the same polynucleotide, such as DNA following transcription, or mRNA following translation, or post-translational modification, such as enzymatic cleavage).

Accordingly, the related forms of the molecules recognised as indicating a particular biological state with regard to the presence of a neurological disease in a mammal are those that would be viewed as being associated with each other, but possess a degree of variation capable of allowing their detection by means known in the art. Preferably, a related form of a biomarker for the determination of a neurological disease may be in one instance a protein that is present in multiple isoforms. Accordingly, it is preferred that the molecules (first and second for example) are related as isoforms.

A protein isoform, as used in the art, refers to variants of a polypeptide that are encoded by the same gene, but that have differences with regard to particular attributes such as their isoelectric point (pI) or molecular weight (MW), or both. It is further considered that a protein isoform as used herein includes both the expected/wild type polypeptide and any natural variants thereof. Such isoforms can arise due to a difference in their amino acid composition (e.g. as a result of alternative mRNA or pre-mRNA processing, e.g. alternative splicing or limited proteolysis) and in addition, or in the alternative, may arise from differential post-translational modification (e.g., glycosylation, acylation, phosphorylation) or can be metabolically altered (e.g. fragmented). The isoforms may be alone or in combination or complexed with another molecule such as Aβ.

It can be contemplated that a protein isoform may also include polypeptides that possesses similar or identical function(s) as a protein isoform but need not necessarily comprise an amino acid sequence that is similar or identical to the amino acid sequence of the protein isoform, or possess a structure that is similar or identical to that of the protein isoform.

As used herein, an amino acid sequence of a polypeptide is “similar” or related to that of a protein isoform if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the protein isoform; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the protein isoform; or (c) the polypeptide is encoded by a nucleotide sequence that is at least 30% (more preferably, at least 35%, at least 40%, at least 45%, at least 50%, at least 55%, at least 60%, at least 65% at least 70%, at least 75%, at least 80%, at least 85%, at least 90%, at least 95% or at least 99%) identical to the nucleotide sequence encoding the protein isoform. As used herein, a polypeptide with a similar structure to that of a protein isoform refers to a polypeptide that has a similar secondary, tertiary or quaternary structure as that of the protein isoform. The structure of a polypeptide can be determined by methods known to those skilled in the art, including but not limited to, X-ray crystallography, nuclear magnetic resonance, and crystallographic electron microscopy.

Accordingly, it can be contemplated that when multiple related forms of a molecule exist, these may be viewed as being numerous isoforms derived from the same particular parental molecule and/or possess a high degree of similarity to the same parent molecule. Any of the biomarkers provided in the present invention are considered to also include their gene and protein synonyms.

In an example of a manner of determining a ratio of molecules, two individual molecules are quantitated by image analysis and the measurement of the intensity of a particular protein spot from a 2D gel is provided. In such an example, a ratio based on the quantitated levels of the levels of the molecules could be represented as:

(level of molecule 1/level of molecule 2)=ratio of molecules

The ratio of molecule levels obtained from the mammal being investigated can then be compared with the previously determined biomarker defined as being characteristic for mammals diagnosed with the neurological disease to identify a statistically significant relationship ideally between the ratios.

In applying the methods of the present invention to validate a biomarker or to use it as a diagnostic or prognostic, it is considered that a clinical or near clinical determination regarding the presence or nature, of a neurological disease in a mammal can be made based on the level or ratio of the validated biomarker. However, the clinical determination may or may not be conclusive with respect to the definitive diagnosis. A diagnosis would be understood by one skilled in the art to refer to the process of attempting to determine or identify a possible disease or disorder, and to the opinion reached by this process.

Furthermore, in characterising the diagnostic capability of a biomarker one of skill in the art may calculate the diagnostic cut-off for the biomarker. This cut-off may be a value, level or range. The diagnostic cut-off should provide a value level or range that assists in the process of attempting to determine or identify a possible disease or disorder.

For example, the level of a biomarker may be diagnostic for a disease if the level is above the diagnostic cut-off. Alternatively, as would be appreciated by one of skill in the art, the level of a biomarker may be diagnostic for a disease if the level is below the diagnostic cut-off.

The diagnostic cut-off for each potential biomarker can be derived using a number of statistical analysis software programs known to those skilled in the art. As an example common techniques of determining the diagnostic cut-off include determining the mean of normal individuals and using, for example, +/−2 SD and/or ROC analysis with a stipulated sensitivity and specificity value. Typically a sensitivity and specificity greater than 80% is acceptable but this depends on each disease situation. The definition of the diagnostic cut-off may need to be rederived if used in a clinical setting different to that in which the test was developed. To achieve this control individuals are measured to determine the mean+/−SD. As one of skill in the art would appreciate, using +/−2 SD outside or away from the measurement obtained from control individuals can be used to identify individuals outside of the normal range. Individuals outside of the normal range can be considered positive for disease. The values obtained in a new clinical setting would then be compared to the historic values to determine if the old diagnostic criteria are still applicable as judged by a statistical test. Individuals known to have the disease condition would also be included in the analysis. In situations where both the disease and control state samples are available ROC analysis method with a chosen sensitivity and specificity may be chosen, typically 80%, to determine the diagnostic value that indicates disease. The determination of the diagnostic cutoff can also be determined using statistical models that are known to those skilled in the art.

Likelihood ratios are also obtained from receiver operating characteristic (ROC) analysis and is calculated as follows:

Likelihood ratio=sensitivity/(1.0−specificity)

The ratio indicates how many times more likely an individual with a given value is to have the disease. For example if someone has a likelihood ratio of 3 then they are 3 times more likely to have disease than someone with a negative test. Similarly, as applied to the biomarker, a high likelihood ratio would indicate a high likelihood that the marker is a biomarker for neurological diseases.

Similarly, the biomarkers identified by the methods of the present invention can be used in providing assistance in the prognosis of a neurological disease and would be considered to assist in making an assessment of a pre-clinical determination regarding the presence, or nature, of a neurological disease. This would be considered to refer to making a finding that a mammal has a significantly enhanced probability of developing a neurological disease.

It would be contemplated that the biomarkers identified by the methods of the present invention could also be used in combination with other methods of clinical assessment of a neurological disease known in the art in providing a prognostic evaluation of the presence of a neurological disease.

The definitive diagnosis of the disease state of a mammal suspected of possessing a neurological disease can be validated or confirmed if warranted, such as through imaging techniques including, PET and MRI, or for instance with the assistance of diagnostic tools such as PiB when used with PET (otherwise referred to as PiB-PET).

The first and second isolated molecule identified in the sample can be selected from the group comprising Aβ, amyloid precursor protein, any member of the serpin family of proteins, any member of the lipoprotein family, or proteins associated with acute phase inflammation response. However, the second isolated molecule is a related form of the first isolated molecule. Preferably, the first or second isolated molecule identified in the sample from a mammal can be selected from the group comprising antithrombin III, serum amyloid P, apoJ, alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alphatrypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof. Preferably, the isoforms or naturally occurring derivatives thereof are selected from the group comprising isoforms A, B, C or J of antithrombin III, isoforms B, C, D, F, G, H or J of serum amyloid P (SAP), isoforms A, B, C, D, E, F or G of apoJ or isoforms A, B, C, D, E, F, G, H or I of alpha-1-microglobulin. More preferably, the isoforms are selected from the group comprising isoform A, B, or J of ATIII, isoform F, B or J of SAP or isoform A, C, D, E, F or G of apoJ, isoform E or G of alpha-1-microglobulin.

Alternatively, the first or second isolated molecule is complexed with Aβ. In this alternative, preferably, the second isolated molecule is selected from the group comprising antithrombin III, serum amyloid P, apoJ, alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alphatrypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof in conjunction or in complex with Aβ. Preferably, the isoforms or naturally occurring derivatives thereof are selected from the group comprising isoforms A, B, C or J of antithrombin III, isoforms B, C, D, F, G, H or J of serum amyloid P (SAP), isoforms A, B, C, D, E, F or G of apoJ or isoforms A, B, C, D, E, F, G, H or I of alpha-1-microglobulin. More preferably, the isoforms are selected from the group comprising isoform A, B, or J of ATIII, isoform F, B or J of SAP or isoform A, C, D, E, F or G of apoJ, isoform E or G of alpha-1-microglobulin.

In another aspect of the present invention there is provided a biomarker for a neurological disease, said biomarker being capable of diagnosis, differential diagnosis and prognosis of a neurological disease wherein the neurological disease is selected from the group comprising Alzheimer's disease (AD), Parkinson Disease (PD), dementia with Lewy bodies (DLB), multi-infarct dementia (MID), vascular dementia (VD), schizophrenia and/or depression. Preferably, the biomarker is capable of diagnosis, differential diagnosis and prognosis of Alzheimer's disease (AD), or Parkinson Disease (PD). The biomarker may be selected form the group comprising Aβ, amyloid precursor protein, any member of the serpin family of proteins, any member of the lipoprotein family, or proteins associated with acute phase inflammation response. However, the second isolated molecule is a related form of the first isolated molecule. Preferably, the first or second isolated molecule identified in the sample from a mammal can be selected from the group comprising antithrombin III, serum amyloid P, apoJ, alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alphatrypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof. Preferably, the isoforms or naturally occurring derivatives thereof are selected from the group comprising isoforms A, B, C or J of antithrombin III, isoforms B, C, D, F, G, H or J of serum amyloid P (SAP), isoforms A, B, C, D, E, F or G of apoJ or isoforms A, B, C, D, E, F, G, H or I of alpha-1-microglobulin. More preferably, the isoforms are selected from the group comprising isoform A, B, or J of ATIII, isoform F, B or J of SAP or isoform A, C, D, E, F or G of apoJ, isoform E or G of alpha-1-microglobulin.

Most preferably the biomarkers for AD are selected from the group comprising antithrombin III, serum amyloid P, apoJ, alpha-1-microglobulin or their naturally occurring derivatives or isoforms thereof. Most preferably the biomarker for AD is antithrombin III or their naturally occurring derivatives or isoforms thereof. Preferably, the isoforms are B or J of ATIII.

Most preferably the biomarker for PD is alpha-1-microglobulin or their naturally occurring derivatives or isoforms thereof. Preferably the isoform of alpha-1-microglobulin is isoform E or G.

In various embodiments, the sensitivity achieved by a validated biomarker(s) and/or clinical markers identified by the presently claimed method for prognosing or aiding diagnosis of a neurological disease is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In various embodiments, the specificity achieved by the use of the set of biomarkers in a method for prognosis or aiding diagnosis of a neurological disease is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In various embodiments, the overall accuracy achieved from validated biomarkers in a method for prognosing or aiding diagnosis of a neurological disease is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In some embodiments, the sensitivity and/or specificity are measured against a clinical diagnosis of neurological disease.

In validating the first molecule as a biomarker of a neurological disease, a ratio may be generated between the levels of the first and the second molecules. Preferably the ratio is generated between isoforms of the first molecule when the second molecule is a related form of the first. Where the first molecule is selected from the group comprising antithrombin III, serum amyloid P, apoJ, alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alphatrypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof a ratio is generated between isoforms selected from the group comprising isoforms A, B, C or J of antithrombin III, isoforms B, C, D, F, G, H or J of serum amyloid P (SAP), isoforms A, B, C, D, E, F or G of apoJ or isoforms A, B, C, D, E, F, G, H or I of alpha-1-microglobulin. More preferably, the isoforms are selected from the group comprising isoform A, B, or J of ATIII, isoform F, B or J of SAP or isoform A, C, D, E, F or G of apoJ, or isoform E or G of alpha-1-microglobulin.

Preferably, where the first molecule is ATIII, the ratio is generated between at least isoforms A, B, C and J such as but not limited to NJ, B/J or C/J.

Preferably where the first molecule is SAP, the ratio is preferably generated between isoforms F and J,

Preferably, where the first molecule is ApoJ, the ratio is preferably generated between isoforms A, B and D.

Preferably, where the first molecule is alpha-1-microglobulin, the ratio is preferably generated between isoform E or G of alpha-1-microglobulin.

In another aspect of the present invention, there is provided a method for diagnosis, differential diagnosis and/or prognosis of a neurological disease in a patient including:

(a) obtaining a first sample from the patient
(b) isolating and identifying a molecule with heparin binding affinity from the first sample wherein the molecule is validated as a biomarker for the neurological disease as herein described;
(c) determining whether the patient is diagnosed, differentially diagnosed and/or prognosed with the neurological disease based on the level of the molecule identified in step b).

In yet another aspect there is provided a method for diagnosis, differential diagnosis and/or prognosis of a neurological disease in a patient including:

(a) obtaining a sample from the patient;
(b) isolating and identifying at least two related forms of a biomarker validated according to the methods described herein from the sample;
(c) determining a level of the biomarkers from (b);
(d) generating a ratio between the levels of the two related forms of the biomarkers identified in step (b);
(e) concluding from the ratio generated in step (d) whether the mammal is diagnosed, differentially diagnosed and/or prognosed with a neurological disease based on the ratio value compared with a reference ratio.

Accordingly, the present invention further relates to uses of biomarkers and their naturally occurring derivatives and isoforms thereof that have been identified as herein described and can be used to determine whether a mammal will possess or will be likely to develop a disease of a neurological origin or assess the mammal for cognitive deterioration. In particular, the present invention is useful for diagnosis, differential diagnosis and/or prognosis of a neurological disease that has a relationship with the increased presence of amyloid and/or amyloid fragments, such as beta amyloid, in the neocortex of a mammal. More particularly, the present invention provides a method that correlates with measurements obtained from PiB-PET studies or AV-45 measurements.

The methods of the present invention may also be used in a pre-screening or prognostic manner to assess a mammal for a neurological disease, and if warranted, a further definitive diagnosis can be conducted with, for example, PiB-PET. Moreover, the biomarkers identified by the methods of the present invention may be useful for selecting patients for clinical assessment using previously validated diagnostic tests, in particular PiB-PET assessment.

The neurological diseases that may be considered to be of relevance to the present invention are those that would include, but are not specifically limited to, Alzheimer's disease (AD), Parkinson's Disease (PD), dementia with Lewy bodies (DLB), multi-infarct dementia (MID), vascular dementia (VD) and/or depression. A preferred disease that may be diagnosed, differentially diagnosed and/or prognosed through the use of the methods of the present invention is AD or PD.

In applying the methods of the present invention, it is considered that a clinical or near clinical determination regarding the presence or nature, of a neurological disease in a mammal can be made and which may or may not be conclusive with respect to the definitive diagnosis. A diagnosis would be understood by one skilled in the art to refer to the process of attempting to determine or identify a possible disease or disorder, and to the opinion reached by this process.

Similarly, the methods of the present invention can be used in providing assistance in the prognosis of a neurological disease and would be considered to assist in making an assessment of a pre-clinical determination regarding the presence, or nature, of a neurological disease. This would be considered to refer to making a finding that a mammal has a significantly enhanced probability of developing a neurological disease.

It would be understood by one skilled in the art that clinical determinations for the presence of a neurological disease would be considered to relate to assessments that include, but are not necessarily limited to, memory and/or psychological tests, assessment of language impairment and/or other focal cognitive deficits (such as apraxia, acalculia and left-right disorientation), assessment of impaired judgment and general problem-solving difficulties, assessment of personality changes ranging from progressive passivity to marked agitation. It would be contemplated that the methods of the present invention could also be used in combination with other methods of clinical assessment of a neurological disease known in the art in providing a prognostic evaluation of the presence of a neurological disease.

The definitive diagnosis of the disease state of a mammal suspected of possessing a neurological disease can be validated or confirmed if warranted, such as through imaging techniques including, PET and MRI, or for instance with the assistance of diagnostic tools such as PiB when used with PET (otherwise referred to as PiB-PET). Accordingly, the methods of the present invention can be used in a pre-screening or prognostic manner to assess a mammal for a neurological disease, and if warranted, a further definitive diagnosis can be conducted with, for example, PiB-PET.

The present invention is based on the finding that the levels or correlations of particular biomarkers are significantly altered in a sample obtained from a mammal determined as having a neurological disease when compared to the levels or correlations of the same biomarkers in a sample obtained from a mammal that is determined not to possess the same neurological disease.

The mammal examined, diagnosed, differentially diagnosed or prognosed through the methods of the present invention may be a human mammal or a non-human mammal. A non-human mammal may be, but is not necessarily considered limited to, a cow, a pig, a sheep, a goat, a horse, a monkey, a rabbit, a hare, a dog, a cat, a mouse or a rat. In one embodiment, the mammal is a primate. In preferred embodiment the mammal is a human, more preferably the mammal is a human adult.

The biomarkers that are of particular interest in the application of the methods of present invention are related forms of the biomarkers that can be derived from, or are similar to antithrombin III, serum amyloid P, apo J (clusterin), alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alpha_trypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof. Preferably, the isoforms or naturally occurring derivatives thereof are selected from the group comprising isoforms A, B, C or J of antithrombin III, isoforms B, C, D, F, G, H or J of serum amyloid P, isoforms A, C, D, E, F or G of apo J or isoforms A, B, C, D, E, F, G, H or I of alpha-1-microglobulin. Preferably, the proteins and isoforms of antithrombin III, serum amyloid P, apo J (clusterin), alpha-1-microglobulin or their naturally occurring derivatives or isoforms thereof are used in accordance with the methods of the present invention.

In providing an assessment of the presence of a neurological disease a sample is obtained from a mammal for interrogation. A sample as would be understood in the practice of the present invention would generally refer to any source of biological material, for instance body fluids, brain extract, peripheral blood or any other source of biological material that can be obtained for the interrogation of the presence of a biomarker.

This accordingly can include a variety of sample types that can obtained from, for instance, a mammal, and which can be used in a prognostic, diagnostic or monitoring manner. These include, but are not necessarily limited to, blood (including whole blood), blood plasma, blood serum, hemolysate, lymph, synovial fluid, spinal fluid, urine, cerebrospinal fluid, semen, stool, sputum, mucus, amniotic fluid, lacrimal fluid, cyst fluid, sweat gland secretion, bile, milk, tears or saliva. Additional examples of samples that may be interrogated for biomarkers include medium supernatants of culture cells, tissue, bacteria and viruses as well as lysates obtained from cells, tissue, bacteria or viruses. Cells and tissue can be derived from any single-celled or multi-celled organism described above.

Preferably, the sample from which a biomarker is determined in the practice of the present invention is a biological sample obtained from a mammal. In more preferred embodiment of the present invention, the sample is the blood from a mammal.

A blood sample may include, for example, various cell types present in the blood including platelets, lymphocytes, polymorphonuclear cells, macrophages, erythrocytes, and may include whole blood or derivatives of fractions thereof well known to those skilled in the art. Thus, a blood sample can also include various fractionated forms of blood or can include various diluents or detergents added to facilitate storage or processing in a particular assay. Such diluents and detergents are well known to those skilled in the art and include various buffers, preservatives and the like. It is considered that this includes samples that have been manipulated in any way after their procurement, such as by treatment with reagents, solubilisation, or enrichment for certain components (such as for proteins or polynucleotides).

In evaluating a mammal for the presence of a neurological disease using the methods of the present invention, the quantification of the amount of at least one biomarker in a sample from a mammal is required so to obtain a level of that biomarker in the sample.

Prior to determining the level of the biomarker, the sample is processed to identify those molecules acting as biomarkers that have heparin binding affinity as herein described. The inventors have identified that molecules having heparin binding affinity can be measured to diagnose, differentially diagnose or prognose a neurological disease. Once the molecule is identified and determined as a biomarker, as herein described, the biomarker or molecule can be analysed to determine whether the mammal has the neurological disease.

It is generally considered that the level of a particular biomarker is a reference to the amount of a particular biomarker in the interrogated sample. For instance, the level of biomarker may be determined and quantified through a primary measurement technique, such that it may be a direct measurement of the quantity or concentration of the biomarker itself. Accordingly, the quantity of a biomarker can be assessed by detecting the number of particular molecules in a sample from a mammal. The level of the biomarker or molecule can be determined as herein described.

Accordingly, it is considered that the biomarkers associated with a neurological disease may be detected and where possible, quantified, by any method known to those skilled in the art. These methods are described herein.

In another aspect there is provided a method for diagnosis, differential diagnosis and/or prognosis of a neurological disease in a patient including:

(a) obtaining a first sample from a patient
(b) isolating and identifying a level of a first and second biomarker with heparin binding affinity from the first sample, wherein the first and the second biomarkers are related and wherein the first and second biomarkers are validated as a biomarker for the neurological disease as herein described,
(c) generating a ratio between the levels of the first and second isolated molecules to provide a generated ratio,
(d) repeating steps (b)-(c) in a second sample obtained from a control to provide a reference ratio,
   (e) comparing the generated ratio identified in the first sample with the reference ratio identified in the second sample;
   (f) concluding a neurological disease status based on a difference between the generated ratio and the reference ratio.

The capacity to recognise whether a mammal is likely to develop a neurological disease results from the identification by the inventors that the quantification, and the comparison, of the respective levels of at least two particular related forms of biomarkers in a sample can be conducted to give an indication of the neocortical amyloid loading of a mammal.

The biomarkers quantified and compared in the present invention are biomarkers that can be obtained from the same sample. Accordingly, by comparing biomarker levels from the same sample, this simultaneous comparison of at least two related forms of the biomarkers provides that a relative comparison is performed and ensures an internal validation of the biomarker levels. This is viewed as removing aspects such as sample-to-sample variability between levels of biomarkers that can exist between mammals and could be regarded as an internal standardisation of the biomarker levels in the sample.

By comparing the respective levels of the at least two related forms of biomarkers, it is possible to generate a ratio. The ratio may be generated from more than two related forms of biomarkers. They may be generated from at least two, three, four, five six, seven, eight, nine or ten related forms of biomarkers. The ratio that is generated between the particular biomarkers can then be utilised, for instance, to prognostically or diagnostically assess whether the mammal will possesses or will be absent a neurological disease by further comparing against a ratio from a control mammal obtained in a similar manner to provide a reference ratio.

It is considered that the term ‘ratio’ or ‘ratios’ would be understood by one of skill in the art to refer to a relationship between the levels of the evaluated biomarkers and a relationship that explicitly indicates a difference in the relative proportions of the levels of the biomarkers examined. As such, the term ratio or ratios represents the relative or proportional level of one biomarker when compared to the level of a second biomarker.

Accordingly, as an example, the relationship or ratio between the levels of one form of a related biomarker to another related form of the biomarker may be the difference between the levels of a parental form of the biomarker and the level of a subsequent fragment derived from the parent biomarker. In one instance, this may be related to a difference between the total level of a parent biomarker and the level of a cleaved fragment from that parent biomarker, such as in one example, a whole protein and a polypeptide fragment cleaved from it under enzymatic digestion. Preferably, the ratio between the levels of the related forms of the biomarkers could be the relationship between protein isoforms and is a difference between total amount a parent protein and the isoform derived from that parent protein. In a preferred embodiment, the ratio is between isoforms derived from the proteins selected from the group comprising antithrombin III, serum amyloid P, apo J (clusterin), alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alphatrypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain and their naturally occurring derivatives, and the parent proteins antithrombin III, serum amyloid P, apo J (clusterin), alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alphatrypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof. Preferably, the isoforms or naturally occurring derivatives thereof are selected from the group comprising isoforms A, B, C or J of antithrombin III, isoforms B, C, D, F, G, H or J of serum amyloid P (SAP), isoforms A, B, C, D, E, F or G of apoJ or isoforms A, B, C, D, E, F, G, H or I of alpha-1-microglobulin. More preferably, the isoforms are selected from the group comprising isoform A, B, or J of ATIII, isoform F, B or J of SAP or isoform A, C, D, E, F or G of apoJ, isoform E or G of alpha-1-microglobulin.

Preferably, where the molecule is ATIII, the ratio is generated between at least isoforms A, B, C and J such as but not limited to NJ, B/J or C/J.

Preferably where the molecule is SAP, the ratio is preferably generated between isoforms F and J,

Preferably, where the molecule is ApoJ, the ratio is preferably generated between isoforms A, B and D.

Preferably, where the first molecule is alpha-1-microglobulin, the ratio is preferably generated between isoform E or G of alpha-1-microglobulin.

A shift or an alteration in a generated ratio based on measuring the levels of the particular biomarkers would thus be anticipated to occur through a change in the level of one biomarker, such as through an increase or decrease (including total absence) in the level of one of the at least two forms of the biomarkers compared in generating the ratio.

The biomarkers identified in the present invention that can provide a ratio able to discriminate whether a mammal is possessive of a neurological disease were initially identified by evaluating, and then comparing, the ratios that existed between biomarkers in samples obtained from various groups of control mammals. In this, the ratio of various forms of the biomarkers in a sample obtained from a mammal possessing a neurological disease (considered as representing a positive control mammal) are compared to the ratio of the same forms of biomarkers in a sample obtained from a mammal that does not possess a neurological disease (considered as representing a negative control mammal).

An indication that a mammal will have or be likely to develop a neurological disease is based on the assessment of the levels of particular forms of related of biomarkers in samples from mammals with an increased level of neocortical amyloid loading (positive control mammals) when compared to mammals determined not to possess increased levels of neocortical amyloid loading (negative control mammals). This assessment of the differing levels of particular related biomarkers is the basis for the development of prognostic tests, for diagnostic tests and/or for the differential diagnosis for neurological diseases based on theoretical neocortical amyloid loading in mammals.

The assessment of whether a mammal has a neurological disease will be determined by the diagnostic cut-off for the biomarker and the likelihood ratio determined for the marker as described herein.

By determining the ratio between at least two forms of related biomarkers from samples obtained from control mammals, it is possible to generate ratios characteristic of a neurological disease state and provide reference ratios. In quantifying and generating the ratios between the related biomarkers, it is considered possible to obtain a series or a range of ratios that can be indicative of various stages or statuses of a neurological disease depending on the appropriate selection of control mammals from where the samples were initially obtained. Accordingly, ratios obtained from such an evaluation may be regarded as being previously defined ratios that are characteristic for a particular disease state in a mammal. Those skilled in the art will also know how to establish, for a given biomarker ratio, a cut-off value suitable for differentiating mammals suffering from a neurological disease from control mammal.

In determining the ratios for related forms of the biomarkers from samples obtained from control mammals, it would be further understood that this information can go to generate a series of reference levels ranges. These reference level ranges can be characteristic for a particular disease state of a mammal based on the ratio provided from the control mammals. Accordingly, those skilled in the art will understand that a suitable reference range of ratios, or a range characteristic for control mammals or mammals suffering from a neurological disease, can also be provided through the methods of the invention.

Preferably the generation of a ratio for use in a method for the diagnosis and/or prognosis in a mammal of a neurological disease related to neocortical amyloid loading is provided by measuring the level of at least two related forms of a biomarker in a sample from a mammal and determining a ratio of the levels of the biomarkers. In particular, the biomarkers quantified in accordance with the methods of the present invention can be selected from the group comprising antithrombin III, serum amyloid P, apo J (clusterin), alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alphatrypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof. Preferably for AD, the biomarkers are selected from the group comprising antithrombin III, serum amyloid P, and apo J (clusterin) or their naturally occurring derivatives or isoforms thereof. For PD the biomarker may be alpha-1-microglobulin or their naturally occurring derivatives or isoforms thereof. Preferably, the isoforms or naturally occurring derivatives thereof are selected from the group comprising isoforms A, B, C or J of antithrombin III, isoforms B, C, D, F, G, H or J of serum amyloid P (SAP), isoforms A, B, C, D, E, F or G of apoJ or isoforms A, B, C, D, E, F, G, H or I of alpha-1-microglobulin. More preferably, the isoforms are selected from the group comprising isoform A, B, or J of ATIII, isoform F, B or J of SAP or isoform A, C, D, E, F or G of apoJ, isoform E or G of alpha-1-microglobulin.

Preferably, where the molecule is ATIII, the ratio is generated between at least isoforms A, B, C and J such as but not limited to NJ, B/J or C/J.

Preferably where the molecule is SAP, the ratio is preferably generated between isoforms F and J,

Preferably, where the molecule is ApoJ, the ratio is preferably generated between isoforms A, B and D.

Preferably, where the first molecule is alpha-1-microglobulin, the ratio is preferably generated between isoform E or G of alpha-1-microglobulin.

As considered in the present invention, the generation of a ratio comprises measuring the level of at least one biomarker, comparing that level to the level of at least one other related biomarker, and determining the ensuing mathematical relationship. Thus, the biomarker ratio is broadly applicable in various uses as considered in the present invention because the biomarker ratio can provide, for instance, a starting point from which additional examination can be performed or a point in which a cross-reference to an equivalent predetermined ratio. The biomarker ratio, due to the inherent capability to provide a normalization effect when the biomarkers measured are those from the same sample, means that the biomarker ratio is not vulnerable to discrepancies that may exist between individuals.

A reference ratio characterised as being indicative of a neurological disease state of a mammal can be used in the diagnosis or prognosis of a neurological disease in a mammal having an unknown neurological disease state. This can be possible when a sample is taken from the mammal with an unknown neurological disease state and a ratio characteristic of a particular neurological disease or disease state is generated (generated ratio). Accordingly, this generated ratio from a mammal can be compared to a previously defined ratio (reference ratio) in order to provide an indication of whether the mammal of unknown disease state will possess a disease. Thus, a correlation of the generated ratio from said mammal with one that is a previously defined ratio (reference ratio) from a control mammal will indicate a likely disease status.

The ratio of biomarker levels obtained from the mammal being investigated can then be compared with the previously determined reference ratio range based on the control to reach a diagnostic or prognostic evaluation of the disease status of the mammal being investigated. The ratio obtained for the mammal under prognosis or diagnosis can also then be compared with this reference range of ratios and, based on this comparison, a conclusion can be drawn as to which neurological disease the mammal is suffering from.

Based on previously determined ratios (reference ratios) of biomarkers from control mammals possessive of a neurological disease state, the ratio between biomarkers may also be used to aid in predicting the amount of neocortical amyloid present in the mammal. Accordingly, the biomarker ratio in a sample from a mammal could also be compared to a range of previously determined ratios in order to extrapolate an expected of level of neocortical amyloid loading in the mammal of interest. The extrapolated levels of neocortical amyloid loading based on the ratios of biomarkers present in the sample from the mammal can accordingly classify the neurological disease state of the mammal relative to a ratio obtained for diagnosed control mammals.

Preferably the generation of a ratio for the assessment of the presence or absence of a neurological disease in a mammal occurs through the quantification of the levels of the proteins and isoforms derived from the proteins selected from the group comprising antithrombin III, serum amyloid P, apo J (clusterin), alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alpha_trypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain and their naturally occurring derivatives or fragments thereof alone or in combination, in samples obtained from mammals. In a particularly preferred embodiment, the generation of a ratio based on the levels of proteins and isoforms of antithrombin III, serum amyloid P, apo J (clusterin), alpha-1-microglobulin or their naturally occurring derivatives or isoforms thereof can be used to diagnose and/or prognose whether a mammal will possess a neurological disease. Preferably, the isoforms or naturally occurring derivatives thereof are selected from the group comprising isoforms A, B, C or J of antithrombin III, isoforms B, C, D, F, G, H or J of serum amyloid P (SAP), isoforms A, B, C, D, E, F or G of apoJ or isoforms A, B, C, D, E, F, G, H or I of alpha-1-microglobulin. More preferably, the isoforms are selected from the group comprising isoform A, B, or J of ATIII, isoform F, B or J of SAP or isoform A, C, D, E, F or G of apoJ, isoform E or G of alpha-1-microglobulin.

Preferably, where the molecule is ATIII, the ratio is generated between at least isoforms A, B, C and J such as but not limited to NJ, B/J or C/J.

Preferably where the molecule is SAP, the ratio is preferably generated between isoforms F and J,

Preferably, where the molecule is ApoJ, the ratio is preferably generated between isoforms A, B and D.

Preferably, where the first molecule is alpha-1-microglobulin, the ratio is preferably generated between isoform E or G of alpha-1-microglobulin.

In the methods of the present invention, at least two biomarkers associated with one or more neurological diseases including antithrombin III, serum amyloid P, apo J (clusterin), alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alphatrypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof are quantified in the generation of a ratio to indicate a neurological disease state of a mammal. It is considered that the predictive power by the simultaneous assessment of the two biomarkers may be improved by adding add least one further biomarker. Detection of an appropriate combination of more than two biomarkers will often increase the specificity and sensitivity of the method. Therefore, it is considered that a combination of at least 2, at least 3, at least 4, at least 5, at least 6, at least 7, at least 8, at least 9, or at least 10 biomarkers can detected in the method of the invention.

Accordingly, in any of the above methods, detection of at least two biomarkers may optionally be combined with detection of one or more additional known biomarkers for neurological diseases, including but not limited to amyloid β peptides, tau, phospho-tau, synuclein, Rab3a, and neural thread protein to improve the predictive assessment that a mammal will possess a neurological disease.

As will be understood in the practice of the methods of the present invention, the evaluation of a prognosis of a neurological disease may vary, and may improve if the sensitivity can be increased. Conventional prognosis of a neurological disease can be determined or confirmed according to any one or more known clinical standards such as the clinical neuropsychology or behaviour assessments as known and recognised and used by health professionals.

It is contemplated therefore that following the quantification of the levels at least two related forms of biomarkers, an additional biomarker may be added which could potentially improve the specificity of determining a neurological disease in a mammal. For instance, the predictive or diagnostic ability of the present invention could be improved by including additional data obtained from further clinical marker values of mammals such as CDR (Clinical Dementia Rating) or Body Mass Index.

Accordingly, the methods of the present invention further consider comparing a ratio of at least two related forms of the biomarkers as herein described and may further include clinical marker values of individuals such as CDR or Body Mass Index from which the set of biological samples was obtained.

In various embodiments, the sensitivity achieved by the use of the set of biomarkers and/or clinical markers in a method for prognosing or aiding diagnosis of a neurological disease is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In various embodiments, the specificity achieved by the use of the set of biomarkers in a method for prognosis or aiding diagnosis of a neurological disease is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In various embodiments, the overall accuracy achieved by the use of the set of biomarkers in a method for prognosing or aiding diagnosis of a neurological disease is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In some embodiments, the sensitivity and/or specificity are measured against a clinical diagnosis of neurological disease.

In a further aspect of the present invention there is provided a method for monitoring the progression of a neurological disease in a mammal, said method comprising

(a) quantifying in a further sample obtained from a mammal previously evaluated for a neurological disease, levels of at least two related forms of a biomarker with heparin binding affinity that were previously evaluated in the mammal;
(b) generating a ratio between the levels of the at least two forms of the related biomarkers in step (a) to provide a generated ratio;
(c) comparing the generated ratio of step (b) with a reference ratio previously defined as characteristic for mammals diagnosed with a neurological disease; wherein the reference ratio is generated following quantifying the levels of the same related biomarkers of step (a) in a sample obtained from at least one control mammal, where at least one control mammal can be positive or negative for the neurological disease; and
(d) concluding from the comparison in step (c) whether the neurological disease status of the mammal previously evaluated for a neurological disease has changed by correlating the generated ratio of step (b) to the reference ratio in a range previously defined as characteristic for the neurological disease for the at least one control mammal.

The changes in the levels of any one or more biomarkers can additionally be used in determining a ratio that may be useful for assessing for any changes in neocortical amyloid loading of a mammal. Accordingly, in the monitoring of the levels of biomarkers in a sample from a mammal, it is possible to monitor for the presence of a neurological disease in a mammal over a period of time, or to track disease progression in a mammal.

Accordingly, changes in the level of any one or more of these biomarkers from a biological sample from a mammal can be used to assess cognitive function, to diagnose or aid in the prognosis or diagnosis of a neurological disease and/or to monitor a neurological disease in a patient (e.g., tracking disease progression in a mammal and/or tracking the effect of medical or surgical therapy in the mammal).

It would be contemplated that an altered level of a biomarker would relate to the appearance or disappearance of the biomarker under examination or to the increase or the decrease of the biomarker under examination in mammals with a certain neurological disease relative to control mammals. Further, it may be contemplated to also relate to an altered level relative to a sample previously taken for the same mammal.

It is contemplated that levels for biomarkers can also be obtained from a mammal at more than one time point. Such serial sampling would be considered feasible through the methods of the present invention related to monitoring progression of a neurological disease in a mammal. Serial sampling can be performed on any desired timeline, such as monthly, quarterly (i.e., every three months), semi-annually, annually, biennially, or less frequently. The comparison between the measured levels and predetermined ratio may be carried out each time a new sample is measured, or the data relating to levels may be held for less frequent analysis.

In a further aspect of the present invention there is provided a method for stratifying or identifying a mammal at risk of developing a neurological disease, said method comprising

(a) quantifying in a sample obtained from a mammal, levels of at least two related forms of a biomarker with heparin binding affinity as herein described;
(b) generating a ratio between the levels of the at least two related forms of the biomarkers in step (a) to provide a generated ratio;
(c) comparing the ratio of step b) with a reference ratio previously defined as characteristic for mammals diagnosed with a neurological disease; wherein the reference ratio is generated following quantifying the levels of the same related biomarkers of step (a) in a sample obtained from at least one control mammal;
(d) concluding from the comparison in step c) whether the mammal is diagnosed, differentially diagnosed and/or prognosed with a neurological disease by correlating the generated ratio of step b) to the reference ratio in a range previously defined as characteristic for the neurological disease for the at least one control mammal; and
(e) based on the conclusion of step d) sorting the mammal into a different classes of the neurological disease based on the severity of the neurological disease differentially diagnosed and/or prognosed in the mammal.

The changes in the level of any one or more of the forms of related biomarkers can accordingly be used to stratify a mammal (i.e., sorting a mammal with a probable diagnosis of a neurological disease or diagnosed with a neurological disease into different classes of the disease). It is considered that the stratifying of a mammal typically refers to sorting of a mammal into a different classes or strata based on the features characteristic of a neurological disease. For example, stratifying a population of mammals with a neurological disease involves assigning the mammals on the basis of the severity of the disease.

Further, the assessment in the change of the levels of any one or more of related biomarkers can be used as a manner of identifying a mammal that may be at risk of developing a neurological disease. It would be considered that should a mammal be identified as being likely to develop a neurological disease, they may be further considered for potential therapeutic intervention to assess if the predisposition of developing a neurological disease can be arrested or attenuated. The effectiveness of the intervention in the progression or development of the neurological disease may be made possible through the monitoring for the change in the ratio between related biomarkers used to generate a ratio indicative of a neurological disease state.

The methods of the invention can additionally be used for monitoring the effect of therapy administered to a mammal, also called therapeutic monitoring, and patient management. Changes in the level of the biomarkers as identified above and associated with one or more neurological diseases, can also be used to evaluate the response of a mammal to drug treatment. In this way, new treatment regimens can also be developed by examining the levels and ratios of the biomarkers in a mammal following commencement of treatment.

In a further aspect, the present invention provides methods for screening for agents that interact with and/or modulate the expression or activity of a biomarker associated with a neurological disease, said method comprising:

(a) contacting a biomarker or a portion of the biomarker with heparin binding affinity as herein described with an agent;
(b) quantifying levels of at least two related forms of the biomarker;
(c) generating a ratio between the levels of the at least two related forms of the biomarkers in step (b) to provide a generated ratio;
(d) comparing the ratio of step c) with a reference ratio previously defined as characteristic for the biomarker in the absence of the agent; wherein the reference ratio is generated following quantifying the levels of the same related biomarkers of step (b) in the absence of the agent;
(e) concluding from the comparison in step d) whether or not the agent interacts with and/or modulates the expression or activity of a biomarker associated with a neurological disease by correlating the generated ratio of step c) to the reference ratio in a range previously defined as characteristic for the biomarker.

It is contemplated that an agent which may be viewed as a potential therapeutic molecule, can include, but is not necessarily be limited to, nucleic acids (DNA or RNA), carbohydrates, lipids, proteins, peptides, small molecules and other drugs. An agent can also be obtained using any of the numerous suitable approaches in combinatorial library methods known in the art, including: biological libraries, spatially addressable parallel solid phase or solution phase libraries, or synthetic library methods. Library compounds for instance may be presented in solution, on beads, chips, bacteria, spores, plasmids, or phage.

The changes in level of any one or more biomarkers that have an influence on the generated ratio may also be evaluated as a manner of tracking the effect of medical or surgical therapy or of the efficacy of therapeutic drug intervention in seeking to a treat neurological disease.

The method of the present invention can thus assist in monitoring a clinical study, for example, for evaluation of a certain therapy for a neurological disease. For example, a chemical compound can be tested for its ability to normalise the level of a biomarker in a mammal having a neurological disease to levels found in control mammals. In a treated mammal, a chemical compound can be tested for its ability to maintain the biomarkers at a level at or near the level seen in control mammals.

In a further aspect of the present invention, there is provided an implementation of the methods as described herein in the form of a system, such as for example, a computer software program, which can be utilised by physicians and researchers to characterise and/or quantify a neurological disease in a mammal.

Accordingly, there is provided for an implementation of the methods as described herein in the form of a system, such as for example, a computer software program, which can be utilised by physicians and researchers to characterise and/or quantify a neurological disease for a subject or a group of subjects.

It is considered that the methods of the invention for assessing whether a mammal will develop a neurological disease may be implemented using any device capable of implementing the aforementioned described methods. Examples of devices that may be used include, but are not necessarily limited to, electronic computational devices, including computers of all types. When the methods described in this application are implemented in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods may also be provided over an electronic network, for example, over the internet, World Wide Web, an intranet, or other network.

In one example, the methods as described herein may be implemented in a system comprising a processor and a computer readable medium that includes program code means for causing the system to carry out the steps of the methods described in this application. The processor may be any processor capable of carrying out the operations needed for implementation of the methods. The program code means may be any code that when implemented in the system can cause the system to carry out the steps of the methods described in this application. Examples of program code means include but are not limited to instructions to carry out the methods described in this application written in a high level computer language such as C++, Java, or Fortran; instructions to carry out the methods described in this application written in a low level computer language such as assembly language; or instructions to carry out the methods described in this application in a computer executable form such as compiled and linked machine language.

Data generated by detection of relevant biomarkers can be analysed with the use of a programmable digital computer. The computer program analyses the data to indicate the number of biomarkers detected, and optionally the strength or level of the signal and the determined molecular mass for each biomarker detected. Data analysis can include steps of determining signal strength or level of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

Analysis of the biomarker levels may further involve comparing the levels of at least two biomarkers with that of a predetermined predictive ratio or a set of relevant values or ratios. In one embodiment, the set of relevant ratios is obtained according to the methods as herein described. Classification analyses or algorithms can be readily applied to analysis of biomarker levels using a computer process. For example, a reference 3D contour plot can be generated that reflects the biomarker levels as described herein that correlate with a disease classification of a neurological disease. For any given mammal, a comparable 3D plot can be generated and the plot compared to the reference 3D plot to determine whether the subject has a biomarker ratio indicative of a neurological disease. Classification analysis, such as classification tree analyses are well-suited for analysing biomarker levels because they are especially amenable to graphical display and are easy to interpret. It will however be understood that any computer-based application can be used that compares multiple biomarker levels from different mammals, or from a reference sample and a mammal, and provides an output that indicates a disease classification of mammal as described herein. The computer can transform the resulting data into various formats for display.

It is also considered that, the ratios of biomarkers indicative of a neurological disease state in a mammal and derived from control mammals can also be inputted into a system to generating a model for predicting the level of neocortical amyloid loading in mammal. Accordingly, a theoretical value for the neocortical amyloid load in a mammal can be determined so to assist in predicting the status or likely status of a neurological disease in said mammal.

The power of a diagnostic or a prognostic model or test to correctly predict status is commonly measured as the sensitivity of the assay, the specificity of the assay or the area under a ROC (Receiver Operating Characteristic) curve. Sensitivity is the percentage of true positives that are predicted by a test to be positive, while specificity is the percentage of true negatives that are predicted by a test to be negative. An ROC curve provides the sensitivity of a test as a function of specificity. The greater the area under the ROC curve, the more powerful the predictive value of the test. Other useful measures of the utility of a test are positive predictive value and negative predictive value. Positive predictive value is the percentage of actual positives who test as positive. Negative predictive value is the percentage of actual negatives that test as negative.

The ROC method has been primarily used as a tool for the measurement of accuracy to define a criterion by which a certain markers can correctly classify a person into a designated class. ROC analyses provides multiple outcomes, one of which, the Area Under the Curve (AUC) is a useful measure for assessing model performance.

The presence or absence of a neurological disease can accordingly also be determined by obtaining a level of at least two forms of a related biomarker in a sample and then submitting the values to statistical analysis by inputting the value in the generated model and obtaining a predictive neocortical amyloid load. The predicted neocortical amyloid load can then associate the subject with the particular risk level of a neurological disease based on the whether the predicted neocortical amyloid load is, for instance, high or low.

In an example of the application of a system utilising the methods of the present invention, for each subject, the information regarding the mammal (e.g. age, gender) is inputted in combination with the quantified levels of at least two related forms of a biomarker. Alternatively, a sample from the mammal being assayed is provided to the system where the system is capable of conducting the measurements and quantification of the levels of two related forms of a biomarker from an individual. The software can then compute a score based on the quantified levels of the two related biomarkers from a mammal in comparison with a predefined ratio that is defined as characteristic of a mammal diagnosed with a neurological disease.

In a further example of this system, the system can return a theoretical amyloid loading for the mammal being assayed and it may also return with an indication that the mammal is either PiB positive or PiB negative by comparing the theoretical amyloid loading of the assayed mammal to that of a reference level from a control mammal in which the PiB status has been previously performed.

The scoring or PiB positive or PiB negative status can then be used either to help in further diagnosing the diseases state of the mammal, to assess the efficacy of a treatment (the score should go down if the treatment is effective), or to compute the average score of a group of mammals in order to study a new therapy or a specific characteristic of the group (e.g. genetic mutation).

In a further example, the efficacy of treatment may be assessed by the reduction of the SUVR score measured on a particular subject. This reduction in the SUVR score would be understood by one of skill in the art to reflect the progression of the mammal towards a neurological disease. It provides a quantitative or close to quantitative assessment of a mammal at a single time point, and allows monitoring the disease progression on a given subject, or a population.

The amyloid loading in a mammal may also be related to the PiB scores obtained by comparison of the ratio generated from two related forms of a biomarker from said mammal when compared to a reference ratio. The amyloid loading can further be understood by one of skill in the art to be normalised to SUVR scores. In a further example, the SUVR score may be either greater than or less than a pre-determined value and which may indicate the likely status of a neurological disease in the assayed mammal based on the calculated neocortical amyloid loading and which is based on the measured reference ratios from control mammals obtained by comparison of biomarkers from biological samples from the control mammals.

In such an example, a SUVR score of less than a pre-determined value corresponds to a healthy person and SUVR score of the pre-determined value or higher may correspond to a person considered to be likely to have or to will likely develop a neurological disease. In a further example of this, the SUVR score may also take into account the demographics of the subject such as age, gender, etc. In yet a further example, it may be conceivable that the threshold SUVR may be lower depending on the appropriate circumstances for measurement or transformation of data.

Accordingly, the methods of the present invention can be applied in a system for monitoring progression of a neurological disease in a mammal through quantitating the levels of at least two related forms of a biomarker from a sample from a mammal, obtaining a ratio between the two related forms of a biomarker and comparing these in the system with predefined ratios from control mammals or a reference ratio generated from samples with known neurological status. For example, a decrease or increase in the ratio from a mammal thereof indicates or suggests progression (e.g., an increase in the severity) of a neurological disease in the mammal. In one example, the monitoring of the neurological disease status of a mammal may be monitored through measurement of the values of the two related forms of a biomarker to determine if the neurological disease status as ascertained by actual, predicted or theoretical SUVR scores, such as changes from greater than the SUVR (indicating a likely positive neurological disease status) to less than the SUVR (indicating a normal or unlikely negative neurological disease status). In a further example, the status of a neurological disease in a mammal may be monitored to determine if the neurological disease status is made worse, such that the neurological disease status changes from less than the SUVR (indicating a normal or unlikely negative neurological disease status), to being greater than the SUVR (indicating a likely positive neurological disease status).

In a further aspect, the present invention provides a kit that can be used for the diagnosis and/or prognosis in a mammal of one or more neurological diseases or for identifying a mammal at risk of developing one or more neurological diseases.

Accordingly, the present invention provides a kit that can be used in accordance with the methods of the present invention for diagnosis or prognosis in a mammal a neurological disease, for identifying a mammal at risk of developing a neurological disease, or for monitoring the effect of therapy administered to a mammal having a neurological disease.

The kit as considered can comprise a panel of reagents, that can include, but are not necessarily limited to, polypeptides, proteins, and/or oligonucleotides that are specific for the biomarkers of the present invention. Accordingly, the reagents of the kit that may be used to determine the level of the biomarkers that are likely to indicate that a subject possesses a neurological disease related to high amyloid loading. For instance, it is envisioned that any antibody that recognises a protein or protein isoform biomarker identified by the methods described herein under examination can be used.

Preferably, a kit for carrying out the methods of the invention comprises a panel of reagents for detecting or monitoring the presence of neocortical amyloid beta loading in an individual, wherein the reagents used are capable of determining the level of at least two forms of a related biomarker for obtaining ratio in accordance with the methods of the invention. Such a diagnostic kit could further be used for the monitoring of the effect of therapy administered to a mammal having a neurological disease.

In a preferred embodiment, the present invention provides a kit of reagents for use in the methods for the screening, diagnosis or prognosis in a mammal of a neurological disease, wherein the kit provides a panel of regents to quantify the level of at least one biomarker in a sample from an mammal, wherein the biomarker is selected from the group comprising antithrombin III, serum amyloid P, apo J (clusterin), alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter alphatrypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof. Preferably, the isoforms or naturally occurring derivatives thereof are selected from the group comprising isoforms A, B, C or J of antithrombin III, isoforms B, C, D, F, G, H or J of serum amyloid P (SAP), isoforms A, B, C, D, E, F or G of apoJ or isoforms A, B, C, D, E, F, G, H or I of alpha-1-microglobulin. More preferably, the isoforms are selected from the group comprising isoform A, B, or J of ATIII, isoform F, B or J of SAP or isoform A, C, D, E, F or G of apoJ, isoform E or G of alpha-1-microglobulin.

It is envisaged that a patient will provide a sample for analysis. The sample may be processed in accordance with the invention and molecules with heparin binding affinity can be isolated and identified in the sample. Preferably, biomarkers selected from the group comprising antithrombin III, serum amyloid P, apo J (clusterin), alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alpha_trypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain, or their naturally occurring derivatives or isoforms thereof can be analysed. A control sample can be processed alongside the patient sample using the same methods. Levels of the biomarkers can be determined and analysed in accordance with the invention. In particular, ratios between isoforms of the biomarkers can be determined. Preferably the ratios will be determined between isoforms of antithrombin III, serum amyloid P, apo J (clusterin), alpha-1-microglobulin, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alpha_trypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof. More preferably the ratios will be determined between the isoforms or naturally occurring derivatives thereof selected from the group comprising isoforms A, B, C or J of antithrombin III, isoforms B, C, D, F, G, H or J of serum amyloid P (SAP), isoforms A, B, C, D, E, F or G of apoJ or isoforms A, B, C, D, E, F, G, H or I of alpha-1-microglobulin. More preferably, the isoforms are selected from the group comprising isoform A, B, or J of ATIII, isoform F, B or J of SAP or isoform A, C, D, E, F or G of apoJ, isoform E or G of alpha-1-microglobulin.

Preferably, where the molecule is ATIII, the ratio is generated between at least isoforms A, B, C and J such as but not limited to NJ, B/J or C/J.

Preferably where the molecule is SAP, the ratio is preferably generated between isoforms F and J,

Preferably, where the molecule is ApoJ, the ratio is preferably generated between isoforms A, B and D.

Preferably, where the first molecule is alpha-1-microglobulin, the ratio is preferably generated between isoform E or G of alpha-1-microglobulin.

A comparison of the generated ratio values of the patient samples compared to the reference samples will enable the diagnosis and/or prognosis in a mammal of one or more neurological diseases or for identifying a mammal at risk of developing one or more neurological diseases.

### EXAMPLES

### Example 1

### Identification and Validation of Biomarkers for Alzheimer's Disease (AD)

### a) Enrichment.

Plasma is one of the most complex matrices available. Thus, it is necessary to reduce the influence of the high abundant proteins that interfere with proteomics analysis. A method of protein enrichment was developed that involves affinity purification using a heparin sepharose column. This technique of protein enrichment removes high abundant proteins such as albumin, haptoglobin IgG and complement C3. The overall enrichment process depletes >90% of the total protein in plasma. The process is reproducible (CV<5%, data not shown) and can be conducted with as little as 10 μL of plasma.

### b) Quantitative 2D Gel Electrophoresis.

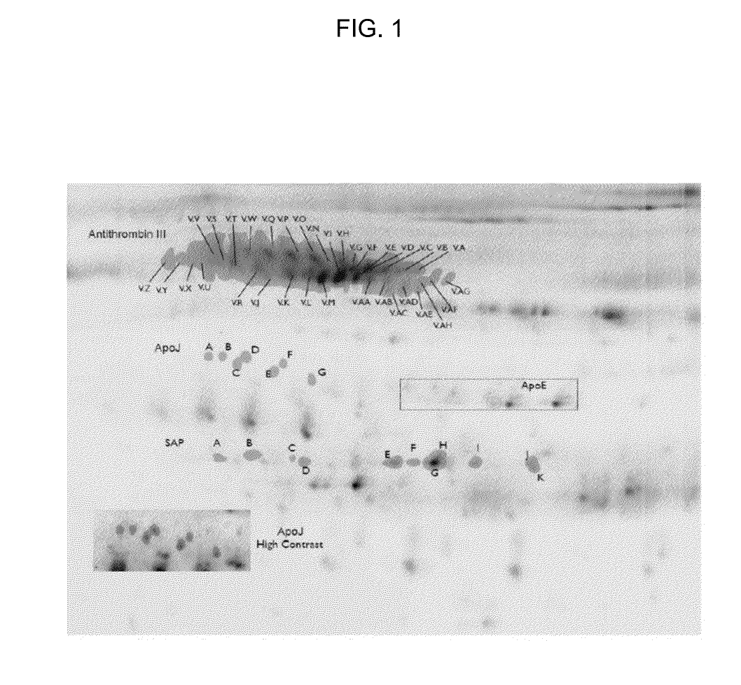

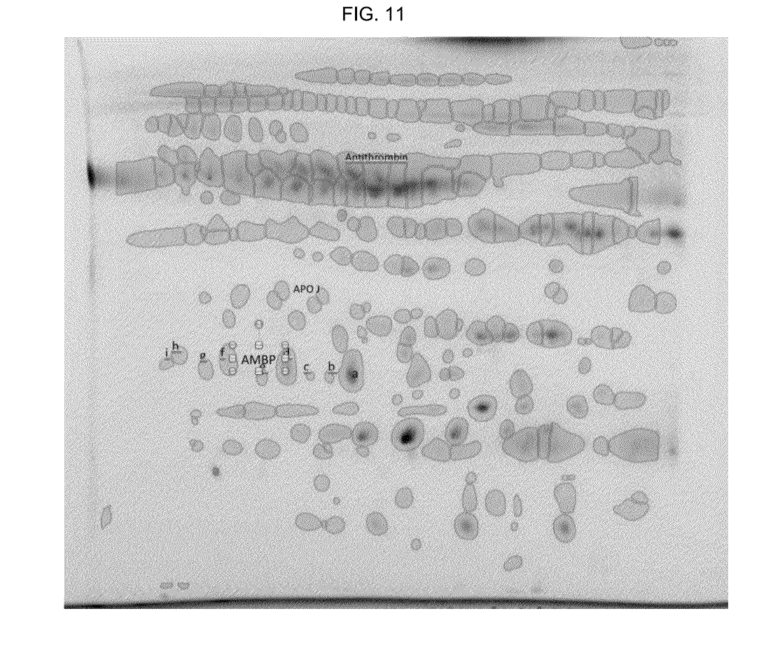

The present example shows that proteins in the blood can reflect the pathological changes that occur in the brain. Specifically, the inventors show that proteins in the plasma of individuals can reflect the amyloid accumulation that occurs in the brain 10-20 years prior to clinical symptoms. By enriching proteins from plasma and utilizing the recently developed Zdyes (provided by Professor Ed Dratz) to perform 2D differential gel electrophoresis the accumulation of the amyloid was shown. A number of different analysis using different isoelectric focusing conditions (pH 3-11 & pH 4-7) were performed and has been shown that using narrow pH range 4.7-5.9 yields the best results for measuring the diagnostic markers; antithrombin III:Aβ, apoJ:Aβ and serum amyloid P in AT patients (FIG. 1) and alpha-1-microglobulin PD patients (FIG. 11).

c) Biomarkers Correlate with Amyloid in the Brain.

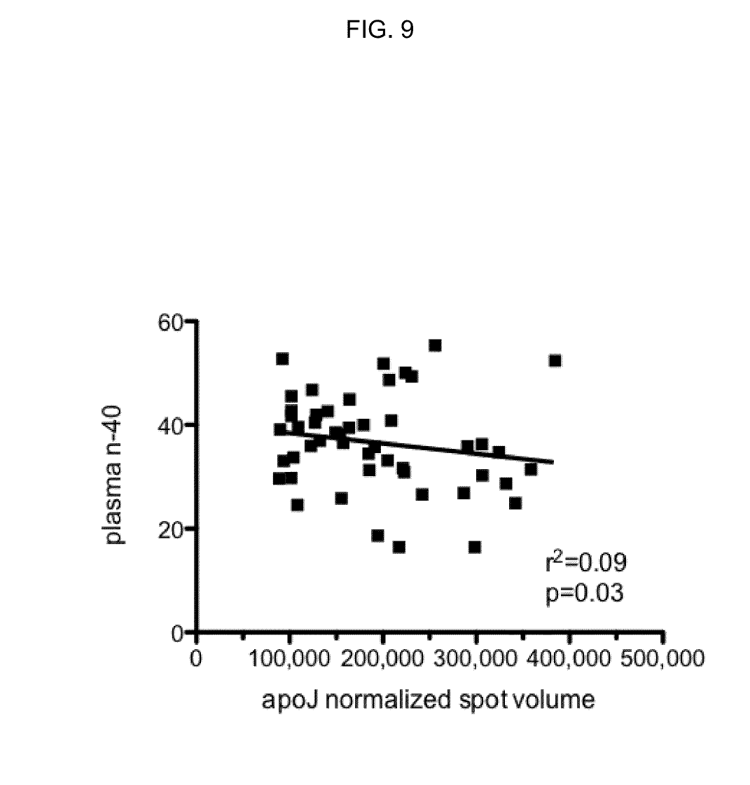

AIBL has one of the largest cohorts of longitudinally PiB-PET imaged individuals. The proteome of 73 individuals from the AIBL baseline cohort with corresponding PiB-PET scan were analysed. The proteomic data was compared to the standard uptake value ratio (SUVR). SUVR is the metric used to determine the retention of PiB in the brain. In this database individuals with a SUVR greater than 1.5 are considered to have high brain-amyloid and prodromal AD. The proteomic analysis yielded over 30 potential biomarkers with greater than a 1.3 fold change and p-value <0.05 by ANOVA after correction for false discovery rate of 5% (manuscript in preparation). The proteins apoJ, antithrombin III and serum amyloid P were the best performing for diagnosis and all had several isoforms with 1.3-2.3 fold changes (p<0.01) between high-amyloid and low-amyloid individuals. The data demonstrates an unprecedented correlation between a plasma biomarker and PiB-PET SUVR (Table 1). Results also show a correlation between ApoJ and neat plasma levels of Aβ (FIG. 9).

### d) Plasma Proteins and Potential Biomarkers.

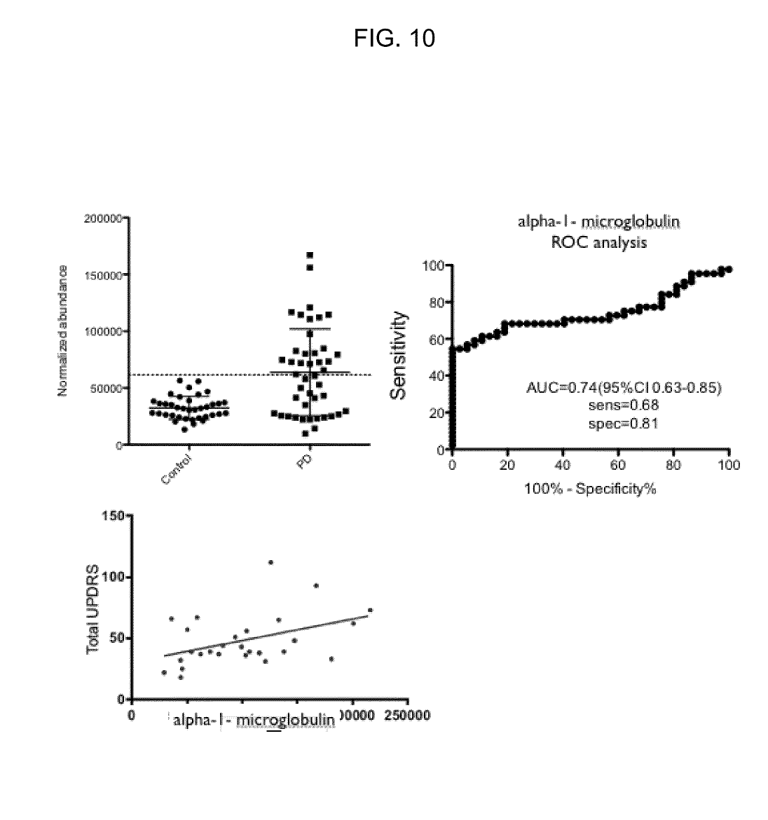

The combination of the heparin sepharose enrichment process, sensitive Zdyes (provided by Al Dratz), and samples from AIBL enabled elucidation of proteins with diagnostic value including antithrombin III, apolipoprotein J (apoJ), and serum amyloid P (Table 1) and alpha-1-microglobulin (FIG. 10).

The proteins were identified using standard protocols for in-gel tryptic digests combined with mass spectrometry (LC-MS/MS, ABSciex 5600 triple TOF & matrix assisted laser desorption time of flight, MALDI-TOF, Bruker Ultraflextreme III). During the process of characterising proteins from the 2D gels it was discovered that gelsolin, actin, antithrombin III, alpha-1-microglobulin and apoJ (a.k.a. clusterin) have isoforms complexed with Aβ. Aβ was sequenced directly using mass spectrometry, Mascot scores for Aβ ranged from 135-330. A Mascot score above 40 indicates positive identification. The presence of Aβ with these proteins has been verified on two independent occasions. Importantly, the diagnostic markers, apoJ and antithrombin III both are found complexed with Aβ. This is consistent with these proteins being involved in the clearance of Aβ. In addition, the presence of Aβ complexed with other proteins would occlude the Aβ epitope from detection with antibody-based techniques, such as ELISA. This may contribute to the lack of diagnostic utility found by measuring Aβ in plasma. The method of analysis directly measures the Aβ:biomarker complex, which circumvents problems of epitope exclusion

TABLE 1
ROC analysis summary

| | Area | | | | | | | [symbol] | |
|---|---|---|---|---|---|---|---|---|---|
| Selected Isoform of | under the | Sensit- | Speci- | Likelihood | Diagnostic Cutoff | Fold | Anova p- | with PIB- | p-value of |
| Diagnostic Markers | [symbol] | ivity % | ficity % | ratio | value +/− STDEV | Change | value* | [symbol] | correlation |
| Antithrombin III ratios | | | | | | | | | |
| A/J | 0.90 | 93 | 82 | 11.9 | 0.155 +/− 14 | 2.6 | <0.0001 | 0.45 | <0.0001 |
| B/J | 0.90 | 86 | 90 | 8.35 | 0.4808 +/− 51 | 2.9 | <0.0001 | 0.44 | <0.0001 |
| C/J | 0.89 | 84 | 79 | 4.1 | 0.938 +/− 1.03 | 2.6 | <0.0001 | 0.46 | <0.0001 |
| Antithrombin III | | | | | | | | | |
| Isoform_A | 0.88 | 84 | 83 | 4.9 | 3825000 +/− 3645000 | 1.7 | 6.00E−09 | 0.31 | <0.0001 |
| Isoform_B | 0.89 | 84 | 86 | 6.1 | 1285000 +/− 5843000 | 1.7 | 6.00E−10 | 0.35 | <0.0001 |
| Isoform_C | 0.84 | 82 | 76 | 3.4 | 29210000 +/− 11780000 | 1.5 | 2.00E−07 | 0.32 | <0.0001 |
| Isoform_J | 0.84 | 77 | 83 | 4.5 | 23810000 +/− 10310000 | 1.6 | 6.00E−09 | 0.33 | <0.0001 |
| ApoJ | | | | | | | | | |
| Isoform_A | 0.70 | 66 | 62 | 1.7 | 83018 +/− 33833 | 1.3 | 0.008 | 0.04 | 0.11 |
| Isoform_B | 0.63 | 65 | 69 | 2.1 | 105753 +/− 37195 | 1.2 | >0.05 | 0.02 | 0.2 |
| Isoform_C | 0.82 | 74 | 83 | 4.3 | 238691 +/− 116244 | 1.6 | <0.0001 | 0.33 | <0.0001 |
| Isoform_D | 0.84 | 79 | 83 | 4.6 | 185909 +/− 97834 | 1.8 | <0.0001 | 0.45 | <0.0001 |
| Isoform_E | 0.86 | 79 | 83 | 4.6 | 173064 +/− 87740 | 1.7 | <0.0001 | 0.48 | <0.0001 |
| Isoform_F | 0.81 | 77 | 79 | 3.7 | 79396 +/− 32621 | 1.6 | >0.05 | 0.42 | <0.0001 |
| Isoform_G | 0.79 | 70 | 66 | 2.1 | 111869 +/− 52527 | 1.4 | 0.0001 | 0.23 | <0.0001 |
| ApoJ isoform Ratio | | | | | | | | | |
| B/D | 0.8 | 72 | 69 | 2.3 | 0.625 +/− 0.2 | −1.4 | <0.001 | 0.32 | <0.0001 |
| A/D | 0.79 | 81 | 76 | 3.4 | 0.488 +/− 0.17 | −1.3 | <0.05 | 0.25 | <0.0001 |
| Serum Amyloid P | | | | | | | | | |
| Isoform_B | 0.80 | 79 | 72 | 2.9 | 672213 +/− 318585 | −1.7 | <0.001 | 0.23 | <0.0001 |
| Isoform_F | 0.75 | 81 | 65 | 2.2 | 515019 +/− 230702 | −1.5 | <0.01 | 0.17 | <0.001 |
| Average Isoform F and Isoform B | 0.74 | 75 | 68 | 2.4 | 518703 | | | | |
| ApoJ E/Serum Amyloid P | 0.82 | 77 | 82 | 4.3 | 0.56 | | | | |

% Sensitivity = The percentage of corectly identified individuals positive for brain amyloid.
% Specificity = The percentage of correctly identified individuals negative for brain amyloid.
Likelihood ratio indicates the probability of having amyloid in the brain.
*ANOVA with Tukey post test [symbol]
indicates data missing or illegible when filed

### Example 2

### Determining the Relationship Between Plasma Biomarkers (apoJ, Antithrombin III and Serum Amyloid P) and Amyloid Deposition in the Brain and Identifying Potential Biomarkers

It is proposed that proteins in the plasma reflect the amyloid load in the brain and therefore will change as brain amyloid accumulates. Pathologically the process that eventually leads to Alzheimer's disease begins ca. 15 years before any clinical signs occur. The inventors have discovered a protein signature in plasma that reflects the presence of amyloid in the brain. Further, these biomarkers demonstrate an ability to diagnose individuals with high brain amyloid (Table 1).

### A) Collecting and Processing Samples

### (i) Samples

Samples are obtained from participants that are either positive for a neurological disease or controls. Individuals are segregated based on their Pittsburgh compound B (PiB) positron emission topography (PET) standard update value ratio (SUVR, High>1.5<Low) which reflects the amyloid load in the brain.

Whole blood was collected from overnight fasted participants by venepuncture. Samples were inverted several times and incubated on a laboratory orbital shaker for approximately 15 minutes at room temperature prior to plasma preparation. Whole blood was collected in two Sarstedt s-monovette, Ethylenediaminetetraacetic acid (EDTA) K3E (01.1605.008) 7.5 mL tubes with prostaglandin E1 (PGE1) (Sapphire Biosciences, 33.3 ng/mL) pre-added to the tube (stored at 4° C. prior to use).

The whole blood was then combined into 15 mL polypropylene tubes and spun at 200×g at 20° C. for 10 minutes with no brake. Supernatant (platelet rich plasma) was carefully transferred to a fresh 15 mL tube, leaving a 5 mm margin in the interface to ensure the red blood cell pellet was not disturbed. The platelet rich plasma was then spun at 800×g at 20° C. for 15 minutes with the brake on. The platelet depleted plasma was then aliquotted into 1 mL Nunc cryobank polypropylene tubes (Thermo Scientific) in 0.25 mL aliquots and transferred immediately to a rack on dry ice and then transferred to liquid nitrogen vapour tanks until required for the assays.

ii) Isolating Molecules with a Heparin Binding Affinity.

### Materials:

HiTrap Heparin HP 1 mL (GE Healthcare Life Sciences)

### Buffer A: 50 mM TRIS pH 8.0, 20 mM NaCl

### Buffer B: 50 mM TRIS pH 8.0, 1.5 M NaCl

45 μL of EDTA plasma was mixed with 180 μL of buffer A. 200 μL of the mixture was loaded onto a HiTrap Heparin HP 1 mL column (heparin sepharose column) at 0.5 mL/min. The column was washed with 5 column volumes buffer A (0.5 mL/min). Proteins (analytes) were eluted from the column using a single step gradient to 100% buffer B then washed with 4 column volumes buffer B (increasing gradient in each wash towards final wash of 100% buffer B) (1 mL/min). Material eluted from the column after each wash with Buffer B was collected in a single 1.5-2 mL fraction. Elution of proteins was monitored using absorbance at 280 nm. After the fourth wash of the column with Buffer B, the column was equilibrated with 5 column volumes of buffer A. After equilibration, the next 200 μL sample (45 μL of EDTA plasma mixed with 180 μL of buffer A) was added to the column.

iii) Processing of the Eluted Material

### (a) Reduction, Alkylation and Precipitation

10 mM TCEP (Tris(2-carboxyethyl)phosphine, Pierce bond breaker neutral pH 500 mM) and 20 mM 4-vinyl pyridine (Sigma) was added to the protein fraction eluted from the heparin sepharose column. The protein fraction was then incubated with rocking for 1 hour at room temperature. After incubation, four volumes of cold acetone was added (e.g. 2 mL faction+8 mL cold acetone (Sigma HPLC grade)).

The fraction containing acetone was briefly mixed by inversion and then incubated at −20° C. overnight (16-20 hours). After overnight incubation, the samples were centrifuged at 4° C. in a swing bucket rotor for 30 min at 4° C. The acetone was then decanted and the remaining protein pellet was washed with 0.5-1 mL of acetone. The acetone was then decanted and the pellet was air dried in a laminar flow hood for approximately 15 minutes at room temperature.

25 μL of 8M urea (GEhealthcare) 4% CHAPS (3-[(3-Cholamidopropyl)dimethylammonio]-1-propanesulfonate, Sigma) was then added to the dried protein pellet and the sample was vortexed until the pellet was dissolved. The sample was centrifuged for 5-10 seconds at 2000×g and then stored at −20° C.

### (b) 2D Gel Analysis

The resuspended protein pellet sample was thawed on ice for ˜1 hour and the protein concentration was determined using the Bradford assay following manufactures instructions (Sigma). 20-75 μg of protein was labelled with 0.5 nmoles of amine reactive fluorescent dye (Zdyes or Cydyes) for 30 min at room temperature. The reaction was quenched by adding 50 mM lysine and incubating for 15 min at room temperature. The labelled proteins were then diluted into rehydration buffer (7M urea, 2M thiourea, 2% CHAPS, trace bromophenol blue) containing 0.5% ampholytes (pH4.7-5.9, BioRad).

The diluted sample was loaded onto dry isoelectric focusing strips (24 cm ReadyStrip IPG, BioRad) by passive rehydration overnight at room temperature. The strips were then focused for a total of 90-110 kVh. After focusing the strips were stored at −20C. Frozen strips were then brought to room temperature and equilibrated 2× with 6 M urea, 4% sodium dodecyl sepharose (SDS), 30% glycerol, 50 mM TRIS pH 8.8 (each wash consisted of a 15 minute incubation at room temperature). The strips were then run in the 2nd SDS dimension using large format (24 cm) 11% SDS-polyacrylimide gel electropohoresis until the dye front was at the bottom of the gel.

### (c) Gel Imaging

The gels were imaged using a Typhoon9500 (GE healthcare). Gel images were processed and the abundance of protein spots compared using the program Progenesis (NonLinear dynamics, v4.5) following manufactures instructions. Anova statistical test, was used to determine if there was a significant difference between proteins. A p-value less than 0.05 is considered to be a significant change. To determine what proteins were changed due to amyloid load in the brain as determined by PiB-PET individuals were compared with SUVR above 1.5 versus control individuals with a PiB-PET less than 1.5.

By utilizing the recently developed Zdyes (provided by Associate Investigator Professor Ed Dratz) to perform 2D differential gel electrophoresis the accumulation of the amyloid can be shown. A number of different analysis using different isoelectric focusing conditions (pH 3-11 & pH 4-7) were performed and has been shown that using narrow pH range 4.7-5.9 yields the best results for measuring the diagnostic markers; antithrombin III:Aβ, apoJ:Aβ and serum amyloid P (FIG. 1).

### (d) Correlating the Markers to PiB/PET

The level of proteins that were found to be significantly changed in High PiB-PET versus Low PiB-PET was graphed against an individual's PiB-PET SUVR value to determine if a correlation existed.

### (e) Validating the Markers as a Markers for AD/PD

Markers were determined to be specific for Alzheimer's disease by the analysis of plasma collected as above from Parkinson's patients. Plasma was processed and analysed from 10 PD patients (as set out above—collecting and processing of samples) and compared to healthy controls. If a protein was found to be significantly changed in High PiB-PET AD patients compared with Low PiB-PET and no significant change was observed in High PiB-PET PD patients, the marker was considered specific to AD.

### (f) Validating the Markers as a Markers for AD Against PD Plasma

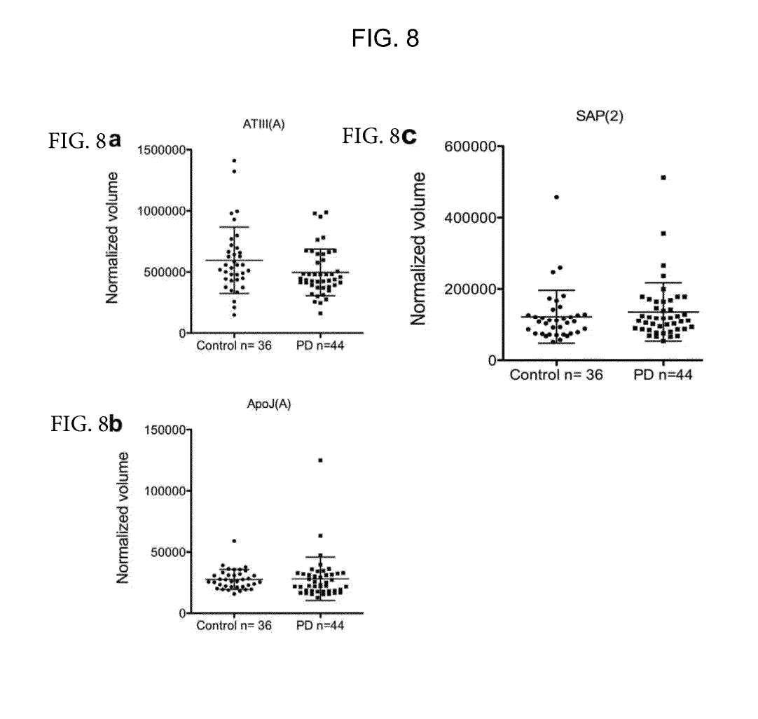

Markers were determined to be specific for Alzheimer's disease by the analysis of plasma collected as above from Parkinson's patients. Plasma was processed and analysed from PD patients (as set out above—collecting and processing of samples) and the markers (ATIII, ApoJ and SAP) from PD plasma were compared. Whilst there were significant changes of these markers in AD plasma, these same AD markers were not elevated in PD plasma (FIG. 8).

### B) Determined Biomarkers to AD.

### (i) Serum Amyloid P (SAP)

Serum amyloid P is a protein known to bind amyloid fibrils and is a universal component of amyloid deposits including AD plaques and neurofibrillary tangles. The data herein demonstrates a negative correlation with PiB-PET-SUVR (Table 1) indicating that the more amyloid in the brain the less Serum amyloid P in plasma, consistent with previous reports.

Comparison of individual SAP isoforms with PiB-PET SUVR also revealed strong, significant correlations with PiB-PET SUVR (Table 1). A ROC curve with 79% sensitivity and 72% specificity was observed for isoform B; 81% sensitivity and 65% specificity was observed for isoform F (Table 1).

Furthermore, a diagnostic intensity cut-off value of 672213+/−318585 was observed for isoform B. Accordingly, individuals with a SAP isoform B spot intensity above 672213 are 2.9 more likely to have AD. A diagnostic intensity cut-off value of 515019+/−230702 was observed for isoform F. Accordingly, individuals with a SAP isoform F spot intensity above 515019 are 2.2 more likely to have AD.

Comparing the ratio between SAP isoforms B and isoform F, revealed a strong significant correlation with PiB-PET SUVR. A ROC curve with 75% sensitivity and 68% specificity was observed (Table 1). Furthermore, a diagnostic cut-off ratio of 518703 was observed. Accordingly, individuals with a SAP isoform ratio above 518703 are 2.4 more likely to have AD (Table 1).

Using the ratio between SAP (isoform B) spot intensity and ApoJ (isoform E) spot intensity, the segregation between AD and controls becomes even more pronounced.

Comparing the ratio between SAP isoform (B) and ApoJ isoform E, revealed a strong significant correlation with PiB-PET SUVR. A ROC curve with 77% sensitivity and 82% specificity was observed (Table 1). Furthermore, a diagnostic cut-off ratio of 0.36 is observed. Accordingly, individuals with a SAP isoform ratio above 0.36 are 4.3 more likely to have AD (Table 1).

### (ii) Antithrombin III (ATIII)

Antithrombin III is the physiological inhibitor of thrombin, an important component of the fibrinolysis and coagulation processes. There has been limited investigation as to the role of antithrombin III and AD. The data herein shows, for the first time, that plasma levels of antithrombin III are elevated in AD and correlate with the deposition of amyloid in the brain (Table 1). It is also shown for the first time that antithrombin III can bind Aβ.

Intensity levels of ATIII isoforms were assessed in samples from high-PiB AD patients and compared with intensity levels of the ATIII isoforms in low-PiB controls. This proteomic analysis identified ATIII (isoform A) as having a 1.7 fold increase in high-PiB AD patients compared with low-PiB controls (p-value=6.00×10⁻⁹) (Table 1). The proteomic analysis also identified ATIII isoforms B (1.7 fold increase; p-value=6.00×10⁻¹⁰), C (1.5 fold increase; p-value=2.00×10⁻⁷) and increased ATIII J (1.6 fold increase; p-value=6.00×10⁻⁹) in high-PiB AD patients compared with low-PiB controls (Table 1).

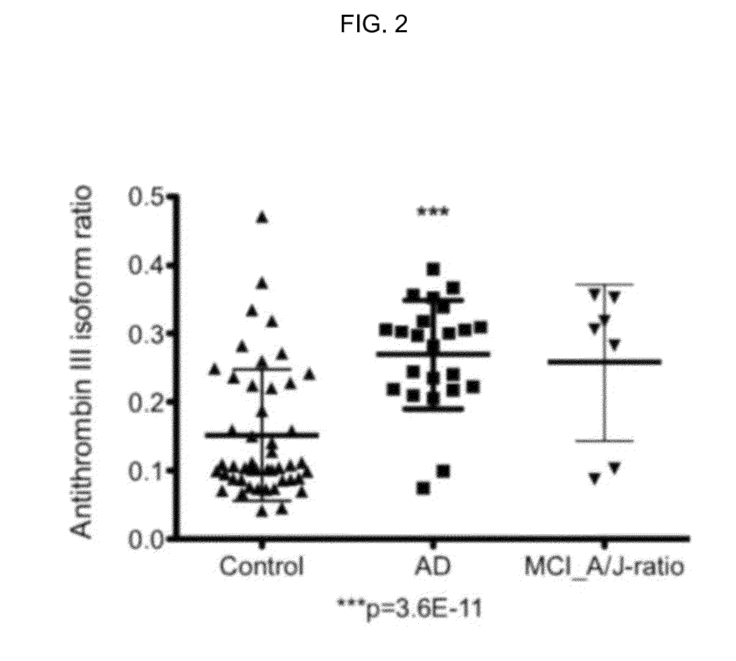

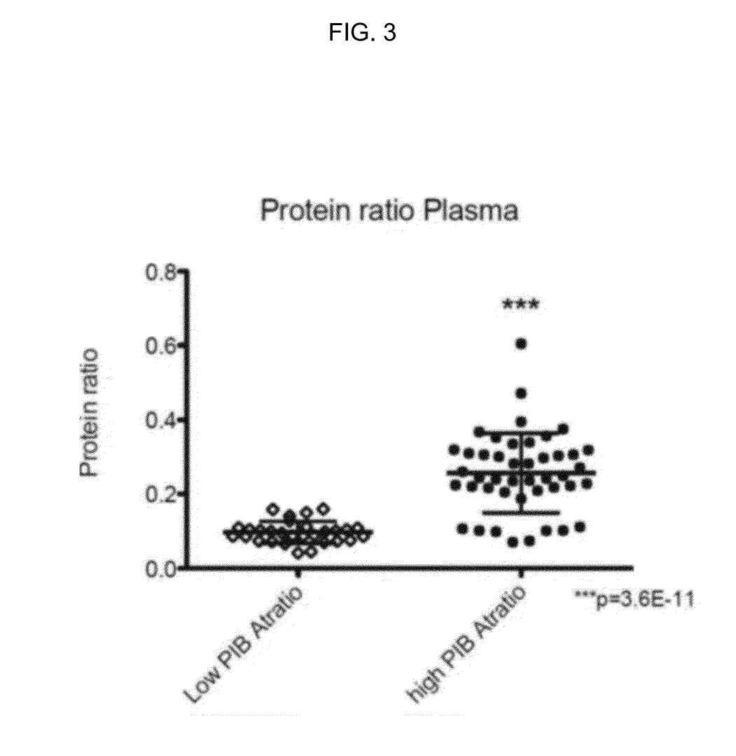

Intensity levels of ATIII isoforms were also compared with the total ATIII spot intensities to obtain a protein expression ratio. This comparison revealed that the ratio of antithrombin III basic isoform to the total ATIII spot intensities was significantly elevated in patients clinically diagnosed with mild cognitive impairment (MCI) and AD compared to cognitively normal individuals (FIGS. 2 and 3).

Using the ratio between the ATIII isoform A protein intensity and ATIII isoform J protein intensity, the segregation between AD and controls becomes even more pronounced (Table 1). The correlation of ATIII (isoform A) and ATIII (Isoform J) alone as a diagnostic is improved as evidenced by the ROC analysis showing an improvement from 0.88 and 0.84 for isoform A and isoform J respectively to 0.9 for the ratio isoform A/J (Table 1).

The correlation of ATIII (isoform B) and ATIII (Isoform J) alone as a diagnostic is also improved as evidenced by the ROC analysis showing an improvement from 0.89 and 0.84 for isoform A and isoform J respectively to 0.9 for the ratio isoform B/J (Table 1).

Similarly, the correlation of ATIII (isoform C) and ATIII (Isoform J) alone as a diagnostic is improved as evidenced by the ROC analysis showing an improvement from 0.84 and 0.84 for isoform C and isoform J respectively to 0.89 for the ratio isoform C/J (Table 1).

Additionally, the correlation of ATIII (isoform A, B and C) and ATIII (Isoform J) alone as a diagnostic is improved as evidenced by the ROC analysis showing an improvement from 0.88, 0.89 and 0.84 for isoform A, B and C and 0.84 for isoform J respectively to 0.8966 for the ratio isoform A, B C/J (Table 1).

(iii) Apo J (Clusterin)

Genome-wide association studies have shown that single nucleotide polymorphisms of the clusterin, the gene that encodes apoJ, are associated with AD However, Silajdzic et al. report that plasma levels of apoJ are not elevated and offer no diagnostic value. The discrepancy in the literature demonstrates the impact that to enrich disease specific proteins will have on our understanding of plasma apoJ in AD. This data show that the diagnostic value of apoJ is captured best in the ROC analysis when apoJ (isoform A, B, C, D and E) is measured (Table 1).

Accordingly the inventors have found three plasma biomarkers that may establish the basis for an early diagnostic test for amyloid accumulation in AD. The work with 2D gels and mass spectrometry has shown that antithrombin III and apoJ (FIG. 9) can be found in plasma, bound to Aβ. PiB-PET imaging reports amyloid burden in the brain.

### Example 3

### Cross-Validate the Accuracy of the Diagnostic Markers Using Independent Samples from the Alzheimer's Disease Neuroimaging Initiative (ADNI, USA)

The biomarkers identified maintain diagnostic accuracy for amyloid in the brain in an independent international cohort. The plasma biomarkers show that they can predict individuals with high amyloid (>1.5 SUVR) in the brain. An important step towards the translation of a diagnostic test into clinical practice is the validation in several international cohorts. As a first step to cross-validating the biomarkers the diagnostic accuracy can be tested in the ADNI study.

ADNI provides 800 PiB-PET imaged individuals and plasma. This tests the validity and robustness of the biomarkers, as the protocols for blood collection and PiB-PET imaging are different from those used by AIBL and the lifestyle and genetic factors of participants are varied compared to the AIBL cohort.

### a) Plasma Samples

These samples can be shipped from ADNI in 6 separate shipments (150 samples/shipment biomarkers can then be extracted) to minimise the risk of losing samples during shipment. The samples were catalogued and stored at −80° C. until analysis. Protein and processed as described above. The samples can be measured with the 2D gel protocol and the MRM-MS assay as above. This allows for the comparison of the 2D gel and MRM-MS results from AIBL directly to those of ADNI.

### b) Statistical Analyses.

The receiver operating characteristic analysis is conducted using Prism v.5.0b. All 2D gel statistical analyses were conducted using Progenesis software (Nonlinear dynamics) and includes correction of false discovery rate and 1-way ANOVA. Further statistical analysis and support was provided by the biostatistician support team that is part of AIBL. The AIBL biostatistics team include modelling variables including age, change in amyloid load, genotype, and clinical neuropsychological metrics.

### Example 4

### Uses of the Diagnostic Test

The clinical use of this diagnostic test could occur as outlined in the following descriptions.

### Scenario 1—Clinical Use

Subjects that are tested for the presence of amyloid in the brain do not need to have symptoms but would likely be in the 6th or 7th decade of life as the presence of amyloid in the brain is present in 10-20% of the population of that age (Rowe et al. 2010, Braak et al. 1996, Sugihara, 1995, Davies 1998). Thus a patient presents to the clinic aged over 60 without clinical symptoms or with cognitive deficits or subjective memory complaints or other deficits in cognitive performance. Blood is collected from the individual using the anti-coagulant EDTA and plasma is recovered for analysis. The analysis is performed using the process described above at Example 2 and one or multiple of the biomarkers are measured. The ratio of specific protein isoforms and the total level of each biomarker are compared to a standard control range. If the test indicates that the individual is positive for the presence of amyloid in the brain, then at several options are available:

a. The individual is referred to confirm the presence of amyloid in the brain via an imaging techniques or cerebral spinal fluid tests.
b. If a viable treatment is available then the individual may have the treatment prescribed.
c. If symptoms exist but the test is negative then other forms of dementia could be tested for.

### Scenario 2—Therapeutic Trials

The accumulation of amyloid begins to occur in the brain 15-20 years before clinical symptoms present (Rowe et al. 2010) and the earlier the disease can be detected the better the chances of preventing the onset of Alzheimer's disease. The biomarker test would then represent a cost effective way to select for individuals with amyloid in the brain to test the efficacy of new therapies.

### Scenario 3—Parkinson's Disease

Individuals that are suspected to have symptoms consistent with Parkinson's disease or other movement disorders would have a blood sample taken using the EDTA as the anticoagulant. The biomarkers present in the plasma would be measured using the process described in this application and the levels of the PD specific biomarkers would be compared to a normal range.

### Scenario 4—Biomarker Discovery

The process described in this application can be applied to discover biological markers of other neurodegenerative diseases. A person wishing to do so would follow the protocol outlined in this application and compare the neurological disease samples to normal controls to determine the appropriate biomarker.

### Example 6

### Deep-Proteomic Screen of Plasma Proteins Reveals Biomarkers for Alzheimer's Disease Using MARS-14 Column—A Comparative Example

The Australian imaging and biomarker lifestyle (AIBL) flagship study of aging was used to search for markers and elucidate mechanisms of AD pathology. Plasma proteins were immuno-depleted and pre-fractionated prior to two-dimensional SDS-PAGE using spectrally resolved fluorescent dyes (Zdyes™) to compare AD and healthy control plasma proteomes. Using recently developed Zdyes, a proteomic screen of intact protein isoforms and their cleavage products was conducted

In this study pooled plasma samples from an initial screen of a sex-matched cohort of N=72 probable sporadic AD patients and N=72 healthy controls were used.

### Materials and Methods

### Immuno-Depletion and Sub-Fractionation.

Three independent pools of ethylenediaminetetraaceticacid plasma were prepared from N=12 subjects for each of male AD (mAD), female AD (fAD), male healthy control (mHC) and female healthy control (fHC) as outlined in Example 2. Pooled plasma samples were immuno-depleted using a multiple affinity removal system (MARS) 14 column (MARS-14, 4.6×100 mm, Agilent) according to manufacturer's instructions. The flow-through, low abundance proteins were collected and fractionated into six sub-fractions using a C18 column (Agilent high-recovery macro-porous 4.6 mm×50 mm).

Sub-fractions were lyophilized and re-suspended for labeling using two spectrally resolved fluorescent dyes (Zdye LLC). Forward and reverse labeling were used to prevent dye bias. Labeled samples were resolved on 24 cm pH 3-11 Immobiline™ Drystrips (GE Healthcare) and 11% acrylamide gels. Gels were scanned for fluorescence using a Typhoon™ Trio scanner (GE Healthcare). False-color images were produced with ImageQuant software (GE Healthcare). Gel image files were imported into Progenesis SameSpots software (Nonlinear Dynamics) for processing, alignment and analysis.

### Identification of Proteins-of-Interest.

To identify changing protein variants, spots-of-interest were excised manually from analytical or preparative gels of fractionated proteins, for in-gel digestion (Sigma-Aldrich proteomics grade porcine trypsin).

### Results

### Deep-Proteomic Investigation of Human Plasma

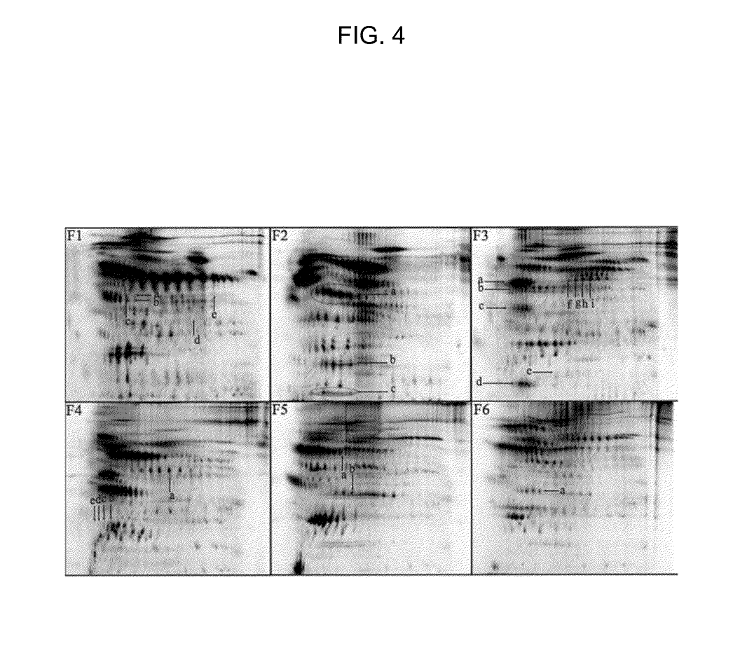

The immuno-depletion and RP sub-fractionation strategy produced six sub-fractions of proteins for comparison by 2DGE. Representative analytical gel images of each of the six RP sub-fractions are shown in FIG. 4. It was estimated that approximately 3,400 unique variants were analyzed by this method, after correcting the total spot count by 10% to account for proteins that eluted in more than one fraction. This is compared to about 610 spots in a gel prepared from a MARS-14 immuno-depleted, but unfractionated plasma. A roughly linear increase in the quantity of protein spots with the number of sub-fractions occurs largely because many co-migrating high MW polypeptides with different hydrophobic characters are separated by RP-HPLC, reducing mutual interference in the analysis of gels. In addition, RP-HPLC enriches proteins, allowing lower abundance species to be more heavily labeled in the covalent protein-dye labeling reactions.

Spots that met the inclusion criteria as described above are listed in Table 3 and are indicated by the arrows in FIG. 4.

Variants, subunits or cleavage products of eight proteins that discriminated AD from control according to the inclusion criteria were identified:

(i) zinc α 2-glycoprotein (ZAG),
(ii) histidine-rich glycoprotein (HRG) fragment,
(iii) haptoglobin (Hot),
(iv) vitamin D binding protein (VDBP),
(v) complement factor I (CFI),
(vi) inter-α trypsin inhibitor (ITHI),
(vii) α-1 anti-trypsin (α1AT) and
(viii) apolipoprotein E (ApoE).

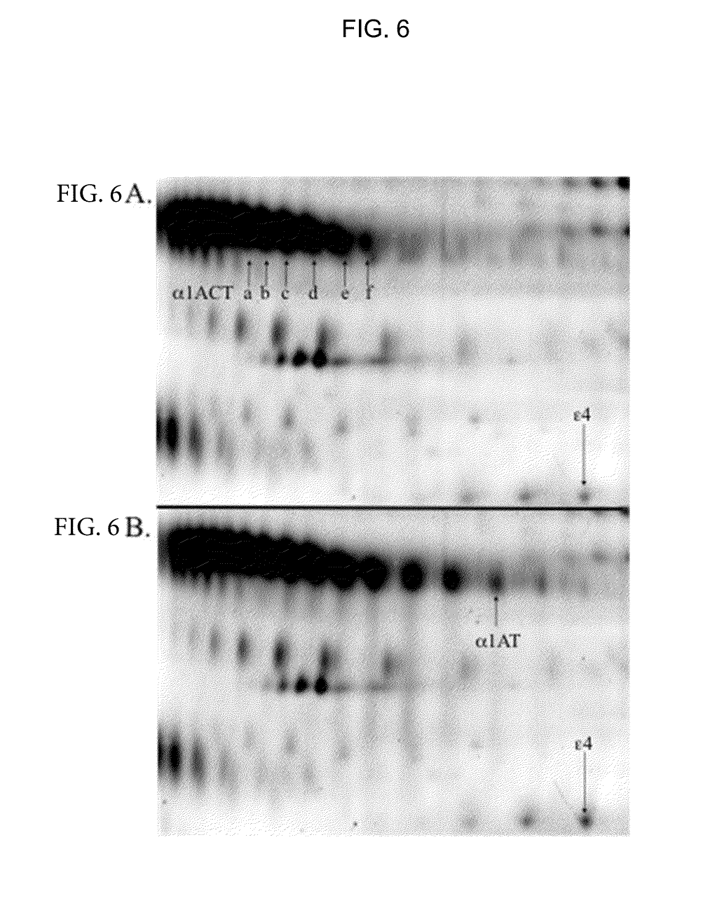

TABLE 2
Potential Marker Analysis

| | | | | Whole | | Male | | | | | | |
|---|---|---|---|---|---|---|---|---|---|---|---|---|
| | | | | cohort | | cohort | | Female cohort | | | | |
| | | RP | | AD | | AD | | AD | | | Mascot | |
| | Acces- | Fract | MW | Fold | | Fold | | Fold | | | score/ | |
| Protein (variant) | sion # | ID | (kDa) | Change | p-val | Change | p-val | Change | p-val | MS | pops | Comment |
| Zinc α 2-glycoprotein | P25311 | 1a | 40 | 1.9 up | <0.05 | NS | NS | NS | NS | 2 | NS | Most basic glycoform |
| (ZAG) | | | | | | | | | | | | |
| Zinc α 2-glycoprotein | P25311 | 1b | 40 | 1.5 up | <0.05 | NS | NS | NS | NS | 2 | III/4 | Most basic glycoform |
| (ZAG) | | | | | | | | | | | | |
| Zinc α 2-glycoprotein | P25311 | 1c | 40 | 1.3 up | 0.05 | NS | NS | NS | NS | 2 | 222/6 | NS but trending |
| (ZAG) | | | | | | | | | | | | |
| Histidine-rich | P04196 | 1d | ~35 | 1.2 up | <0.02 | NS | NS | 2.9 up | <0.002 | 2 | 161/4 | Putative |
| glycoprotein | | | | | | | | | | | | cleavage product |
| (BRG) | | | | | | | | | | | | |
| Unidentified Series | n/a | 1e | ~40 | 1.5 | <0.05 | NS | NS | NS | NS | — | n/a | Low abudance |
| | | | | down | | | | | | | | species |
| Haptoglobin (Hpt) | P00738 | 2a | 40 | 2.6 up | <0.01 | NS | NS | 2.2 up | <0.02 | 2 | 238/13 | Summation of |
| heavy chain | | | | | | | | | | | | varients |
| Haptoglobin (Hpt) mild | P00738 | 2b | 16 | NS | NS | NS | NS | NS | NS | 2 | * | NS changing |
| chain | | | | | | | | | | | | subunit of Hpt |
| Haptoglobin (Hpt) light | P00738 | 2c | 9 | 2.4 up | <0.02 | NS | NS | NS | NS | 2 | * | Summation of |
| chain | | | | | | | | | | | | varients |
| Vitamin D binding | P02774 | 3b | ~50 | NS | NS | See FIG. 1-F3b and FIG. 6A | | | | | | Cleavage products |
| protein (VDBP) | | | | | | | | | | | | |
| Vitamin D binding | P02774 | 3c | ~40 | NS | NS | See FIG. 1-F3c and FIG. 6B | | | | | | Multiple cleavage |
| protein (VDBP) | | | | | | | | | | | | product |
| Vitamin D binding | P02774 | 3d | ~10 | NS | NS | See FIG. 1-F3d and FIG. 6C | | | | | | Multiple cleavage |
| protein (VDBP) | | | | | | | | | | | | product |
| Inter α trypsin | Q14624 | 3e | ~32 | 1.3 up | <0.05 | NS | NS | NS | NS | 3 | 428/5 | C-term product |
| inhibitor heavy chain | | | | | | | | | | | | |
| H4 (IIIH4) | | | | | | | | | | | | |
| Complement factor I | P05156 | 3f | ~53 | NS | NS | 2.5 up | <0.01 | 2.6 down | <0.02 | 2 | 64/2 | Putative cleavage |
| (CFI) | | | | | | | | | | | | product |
| Complement factor I | P05156 | 3g | ~53 | NS | NS | 2.0 up | <0.001 | 2.4 down | <0.03 | 2 | 78/2 | Putative cleavage |
| (CFI) | | | | | | | | | | | | product |
| Complement factor I | P05156 | 3h | ~53 | NS | NS | 3.6 up | 0.056 | 2.1 down | <0.03 | 2 | 127/4 | Putative cleavage |
| (CFI) | | | | | | | | | | | | product |
| Complement factor I | P05156 | 3i | ~53 | NS | NS | 4.0 up | <0.1 | 3.1 down | 0.058 | 2 | 69/1 | NS but |
| (CFI) | | | | | | | | | | | | trending |
| Inter α trypsin | Q14624 | 4a | ~40 | 1.3 up | <0.02 | 1.5 up | <0.02 | NS | NS | 3 | 890/14 | N-term cleavage |
| inhibitor heavy chain | | | | | | | | | | | | |
| H4 (IIIH4) | | | | | | | | | | | | product |
| C-reactive binding | P02741 | 4b | 25 | 3.2 up | <0.02 | NS | NS | NS | NS | 1 | 96/2 | Non- |
| protein (CRP) | | | | | | | | | | | | significant change |
| C-reactive binding | P02741 | 4c | 25 | 2.9 up | 0.39 | NS | NS | NS | NS | 1 | NS | Non- |
| protein (CRP) | | | | | | | | | | | | significant change |
| C-reactive binding | P02741 | 4d | 25 | 2.4 up | 0.18 | NS | NS | NS | NS | 1 | NS | Non- |
| protein (CRP) | | | | | | | | | | | | significant change |
| C-reactive binding | P02741 | 4e | 25 | 2.2 up | 0.06 | NS | NS | NS | NS | 1 | NS | NS but |
| protein (CRP) | | | | | | | | | | | | trending |
| α 1-aztitrypsin (αIAT) | P01009 | 5a | ~47 | 3.3 | <0.02 | NS | NS | NS | NS | 2 | 86/3 | |
| Apolipoprotein E | P02649 | 5b | 34 | 1.5 up | <0.02 | NS | NS | NS | NS | 2 | 149/7 | Epsilon4 epoxy |
| (ApoE) | | | | | | | | | | | | |

This example shows that a different set of biomarkers for AD can be obtained from a process that utilizes MAP-14 to immunodeplete and sub-fractionate the plasma samples compared to the heparin-sepharose columns approach of the present invention. The MARS-14 column targets serum albumin, transferrin, haptoglobin, IgG, IgA, al-antitrypsin, fibrinogen, α2-macroglobulin, α1-acid glycoprotein, complement C3, IgM, apolipoprotein AI, apolipoprotein AII and transthyretin for depletion.

### Example 7

### Biomarker Discovery in Parkinsons Disease—Alpha-1-Microglobulin

All samples were processed as described in the above Examples.

The goal of this study was to apply the biomarker workflow using heparin binding to discover a diagnostic blood based biomarker for Parkinson's disease (PD). The protein alpha-1-microglobulin (AMBP, amino acids 20-203 of the AMBP gene) has been found to be elevated in PD plasma using the heparin sepharose enrichment process as described above. The levels of AMBP are increased with the severity of PD (FIG. 10).

Using 2D gel analysis the markers for alpha-1-microglobulin are apparent (FIG. 11). This figure also shows the various isoforms of alpha-1-microglobulin.

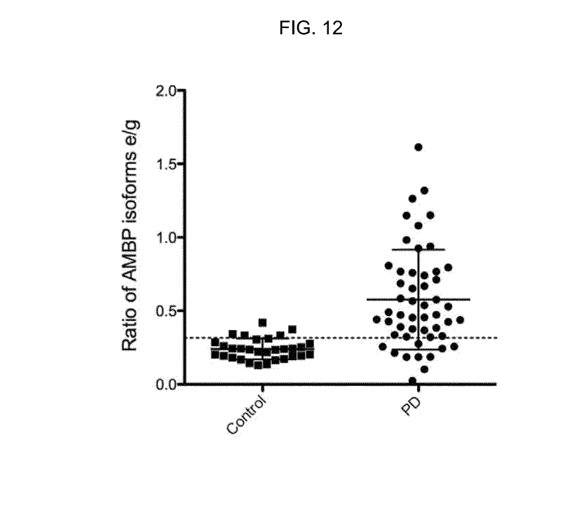

Ratio analysis was conducted as described herein. The ratio of the two isoforms G and E of alpha-1-microglobulin shows that it has better diagnostic accuracy than the isoforms alone (FIG. 10 shows the ROC analysis of isoform E only). ROC analysis results in an area under the curve of 0.86 (95% CI 0.78-0.94) and p value <0.0001. However, the comparison of the ratio of spot numbers or isoforms 193/166 (G/E) between PD and controls is shown in FIG. 12. The dashed line represents 80% specificity of the test and individuals at the cutoff value have a 5.0 odds ratio. (n=31 controls n=51 PD).

### Example 8

### Biomarkers for the Detection of Amyloid in the Brain Before Cognitive Symptoms of Alzheimer's Disease Occurs

### (i) Sample Preparation

EDTA plasma samples were collected as in Example 1 from cognitively normal individuals that were negative for brain amyloid as assessed by PET imaging or positive for brain amyloid. (n=6 negative and n=7 positive). The samples were protein enrichment using a mini spin column of heparin sepharose HP (GE life sciences) consisting of 400 μL of media. Samples were diluted with buffer A as described in Example 2. Proteins were eluted from the heparin sepharose as described in Example 2.

Samples were prepared as in the Examples above. However, the only difference to this point was the use of the mini spin column versus the prepacked columns and an HPLC.

Solid urea was added to the proteins eluted from the heparin sepharose to reach a final concentration of 8M urea. The proteins were reduced with 10 mM dithiothreitol (1 hr 37° C.) and then alkylated with 40 mM iodoacetamide (1 hr 37° C.). The sample was then diluted 8× (e.g. 100 μL sample+700 μL buffer) with 50 mM ammonium bicarbonate pH 8 and proteomics grade trypsin was added at a ratio 1:100 (trypsin:protein) and left to digest overnight at 37° C. The digestion was stopped by the addition of formic acid to a final concentration of 1%. The peptides were then desalted using a C18 solid phase extraction cartridge following manufactures instructions (Waters, 1 cc). The desalted peptides were then concentrated in a centrifugal vacuum concentrator to dryness. Immediately prior to liquid chromatography analysis the peptides were resuspended with 3% acetonitrile in water 0.1% formic acid. 500 ng of peptide was analyzed on a Thermo Scientific Easy-nLC 1000 HPLC system coupled to a QExactive plus.

### (ii) Peptide Separation

The samples were initially loaded onto a Thermo Acclaim PepMap C18 trap reversed-phase column (75 μm×2 cm nanoviper, 3 μm particle size) at a maximum pressure setting of 800 bar. Separation was achieved at 300 nL/minute using buffer A (0.1% formic acid in water) and buffer B (0.1% formic acid in acetonitrile) as mobile phases for gradient elution with a 75 μm×25 cm PepMap RSLC C18 (2 μm particle size) Easy-Spray Column at 35° C.

Peptide elution employed a 3-8% acetonitrile gradient for 10 mins followed by 10-40% acetonitrile gradient for 30 mins. The total acquisition time, including a 95% acetonitrile wash and re-equilibration, was 62 minutes. The eluted peptides from the C18 column were introduced to mass spectrometer via nanoESI, and analysed using the Q-Exactive Plus instrument. (Thermo Fisher Scientific, Waltham, Mass., USA). The electrospray voltage was 1.8 kV, and the ion transfer tube temperature was 320 C. Employing a top 15 data dependent MS2 acquisition method excluding unassigned and +1 charged species, Full MS Scans were acquired in the Orbitrap mass analyzer over the range m/z 400-1600 with a mass resolution of 70 000 (at m/z 200). The target value was 3.00E+06. The 15 most intense peaks with charge state were isolated using an isolation window of 1.4 m/z and fragmented in the HCD collision cell with normalized collision energy of 27%. Tandem mass spectra were acquired in the Orbitrap mass analyzer with a mass resolution of 17,500 at m/z 200. The automatic gain control target value was set to 2.0E+05. The ion selection threshold was set to 2.00E+04 counts. The maximum allowed ion accumulation time was 30 ms for full MS scans and 50 for tandem mass spectra. For all the experiments, the dynamic exclusion time was set to 10 s.

(iii) Peptide Analysis

Database searching was performed with Proteome Discoverer 1.4 (Thermo Fisher Scientific) initially using SEQUEST HT for searching against a non-redundant human database. Database searching against the corresponding reversed database was also performed to evaluate the false discovery rate (FDR) of peptide identification. The SEQUEST HT search parameters included a precursor ion mass tolerance 10 ppm and product ion mass tolerance of 0.08 m/z units. Cysteine carbamidomethylation was set as a fixed modification, while M oxidation, C-terminal amidation and deamidated (of NQ) as well as N-terminal Gln to pyro-Glu were set as variable modifications. For all database searching, Trypsin digestion with up to 2 missed cleavages was specified for the digestion parameters. Differential analysis was undertaken using SEIVE 2.1 (ThermoFisher), with an A vs. B differential experimental model.

### (iv) Results

This comparison between 6 healthy controls negative for brain amyloid (determined by PET imaging) and 7 cognitively normal controls positive for brain amyloid shows that by using the heparin sepharose protein enrichment of the present invention biomarkers have been shown to be elevated due to amyloid load in the brain of healthy controls. Previous literature has focussed on comparing controls with AD patients.

Results are shown in Table 3.

TABLE 3
Biomarkers for brain amyloid discovered using mass spectrometry

| DESCRIPTION | PEPTIDES | Ratio | StdDev | PValue |
|---|---|---|---|---|
| ANT3_HUMAN | 4 | 1.2 | 0.19 | 2.41E−03 |
| Antithrombin_III | | | | |
| APOH_HUMAN | 9 | 1.3 | 0.18 | 8.16E−06 |
| Beta_2_glycoprotein 1 | | | | |
| FIBB_HUMAN | 8 | 1.3 | 0.10 | 2.64E−08 |
| Fibrinogen beta chain | | | | |
| FIBA_HUMAN | 3 | 1.6 | 0.43 | 1.85E−07 |
| Fibrinogen alpha chain | | | | |
| C9JC84_HUMAN | 2 | 1.3 | 0.36 | 1.28E−02 |
| Fibrinogen gamma chain | | | | |
| ITIH2_HUMAN | 7 | 1.3 | 0.25 | 2.23E−04 |
| Inter_alpha_trypsin | | | | |
| inhibitor heavy chain H2 | | | | |
| HRG_HUMAN | 6 | 1.5 | 0.28 | 1.41E−07 |
| Histidine_rich glycoprotein | | | | |
| B0UZ83_HUMAN | 5 | 1.3 | 0.24 | 1.60E−03 |
| Complement C4 beta chain | | | | |
| CFAH_HUMAN | 4 | 1.3 | 0.23 | 2.47E−03 |
| Complement factor H | | | | |
| HEP2_HUMAN | 4 | 1.3 | 0.25 | 3.06E−04 |
| Heparin cofactor 2 | | | | |
| E9PBC5_HUMAN | 2 | 1.7 | 0.71 | 1.52E−03 |
| Plasma kallikrein heavy chain | | | | |

In Table 3 the name of the protein is followed by the number of tryptic peptides that were measured, the change in the abundance of the protein, the standard deviation in the change and p-value from a t-test. The ratio is averaged from the change of each individual peptide that was analysed for the given protein. A ratio greater than 1.0 indicates an increase in protein abundance.

Regarding ratio of proteins; the data shows that these are potentially diagnostic. These potential biomarkers are changed in individuals that have high brain amyloid. Thus the ratio improves the diagnostic potential of the biomarkers and this data from MS can be further analysed using the measurement of a ratio of two peptides from one biomarker such as antithrombin III for example. Using the ratio of two peptides from the same protein would have many advantages for controlling sample storage and handling.

This demonstrates that the use of heparin sepharose in the processing of the samples prior to the separation of proteins or peptides provides access to potentially diagnostic biomarkers which can form the basis of a sensitive diagnostic for AD, even before cognitive symptoms of Alzheimer's disease occurs.

This data shows a different proteomic technique (mass spectrometry (MS)) for discovering biomarkers, some of which are the same between both techniques such as DGE and MS (i.e. antithrombin III). Hence this validates that ATIII shows potential as a diagnostic marker for AD.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as broadly described herein.

What is claimed is:

1. A method of identifying a biomarker of a neurological disease comprising the steps of:
(a) isolating a first molecule with heparin binding affinity from a first sample that is positive for a neurological disease; and
(b) validating the first molecule as a biomarker of the neurological disease against a known marker of a neurological disease.

2. The method of claim 1 wherein validating the isolated molecule as a biomarker comprises the steps of:
(a) identifying a level of the first isolated molecule with heparin binding affinity in the first sample that is positive for a neurological disease;
(b) identifying a level of another biomarker previously defined as being characteristic for mammals diagnosed with the neurological disease present in the first sample;
(c) comparing the level of the isolated molecule identified in step (a) with the level of the other biomarker identified in step (b) to identify a statistically significant relationship between the level of the isolated molecule and the level of the other biomarker;
(d) repeating steps (a)-(c) in a second sample obtained from a control to determine whether the relationship identified in the first sample is identified in the second sample;
(e) concluding that the first isolated molecule is a biomarker of the neurological disease if the relationship identified in the first sample is not identified in the second sample.

3. The method of claim 1 or 2 further comprising the steps of:
(a) isolating and identifying a level of a second molecule with heparin binding affinity from the first sample, the second isolated molecule being related to the first isolated molecule,
(b) generating a ratio between the levels of the first and second isolated molecules,
(c) comparing the ratio generated in step b) with the level of another biomarker previously defined as being characteristic for mammals diagnosed with the neurological disease present in the first sample to identify a statistically significant relationship between the ratio of step b) and the level of the other biomarker,
(d) repeating steps (a)-(c) in a second sample obtained from a control to determine whether the relationship identified in the first sample is identified in the second sample;
(e) concluding that the ratio is a biomarker of the neurological disease if the relationship identified in the first sample is not identified in the second sample.

4. The method of claim 2 or 3 wherein the other biomarker previously defined as being characteristic for mammals diagnosed with the neurological disease is a neocortical amyloid level characteristic of the neurological disease.

5. The method of any one of claims 2 to 4 wherein the other biomarker previously defined as being characteristic for mammals diagnosed with the neurological disease is the level of a radiotracer specifically recognising the presence of the amyloid beta detected in the brain.

6. The method of claim 5 wherein the radiotracer is selected from the group comprising Pitsburg compound B (PiB), florpiramine F-18, Floubetaben, Florbetapir, Flutematamol and NAV4694.

7. The method of any one of claims 2 to 4 wherein the other biomarker is a PiB-PET SUVR score.

8. The method according to any one of claims 1 to 7 wherein the isolated molecule is isolated along with Aβ.

9. The method of any one of claims 1 to 8 wherein the first isolated molecule or the second isolated molecule are isolated by binding the molecules to heparin.

10. The method of any one of claims 1 to 9 wherein the first or second isolated molecule(s) are isolated on a heparin sepharose column.

11. The method of claim 10 wherein the heparin-sepharose column is eluted at about pH 1.0 to about pH 11.0 to isolate molecules.

12. The method of claim 10 wherein the heparin-sepharose column is eluted at about pH 8.0 to isolate molecules.

13. The method of claim 10 wherein the heparin-sepharose column is eluted at about pH 7.0 to isolate molecules.

14. A method according to any one of claims 1 to 13 wherein at least the first and control samples are pretreated to remove high abundant proteins prior to isolating molecules with heparin binding affinity.

15. A method according to any one of claims 2 to 14 wherein the level of the molecule is determined using a technique selected from the group comprising 2D DGE, mass spectrometry (MS) such as multiple reaction monitoring mass spectrometry (MRM-MS), Real Time (RT)-PCR, nucleic acid array; ELISA, functional assay, by enzyme assay, by various immunological methods, or by biochemical methods such as capillary electrophoresis, high performance liquid chromatography (HPLC), thin layer chromatography (TLC), hyper-diffusion chromatography, two-dimensional liquid phase electrophoresis (2-D-LPE) or by their migration pattern in gel electrophoreses and Sodium dodecyl sulfate-polyacrylamide gel electrophoresis (SDS-PAGE).

16. The method according to any one of claims 3 to 15 wherein the second molecule is related as an isoform of the first molecule.

17. The method of any one of claims 1 to 16 wherein the neurological disease is Alzheimer's disease or Parkinson's disease.

18. A biomarker for the diagnosis, differential diagnosis and/or prognosis of a neurological disease selected from the group comprising antithrombin III, serum amyloid P, ApoJ, ANT3_HUMAN Antithrombin_III, APOH_HUMAN Beta_2_glycoprotein, FIBB_HUMAN Fibrinogen beta chain, FIBAHUMAN Fibrinogen alpha chain, C9JC84_HUMAN Fibrinogen gamma chain, ITIH2_HUMAN Inter_alpha_trypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or alpha-1-microglobulin or their naturally occurring derivatives or isoforms thereof.

19. A biomarker identified by the method according to any one of claims 1 to 17 when used for the diagnosis, differential diagnosis and/or prognosis of a neurological disease.

20. A biomarker according to claim 18 or 19 selected from the group comprising antithrombin III, serum amyloid P, ApoJ, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alpha_trypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof wherein the neurological disease is Alzheimer's disease.

21. A biomarker according to claim 18 or 19 which is alpha-1-microglobulin or its naturally occurring derivatives or isoforms thereof wherein the neurological disease is Parkinson's disease.

22. A biomarker according to any one of claims 18 to 21 wherein the isoforms or naturally occurring derivatives thereof are selected from the group comprising isoforms A, B, C or J of antithrombin III, isoforms B, C, D, F, G, H or J of serum amyloid P (SAP), or isoforms A, B, C, D, E, F or G of apoJ, isoforms A, B, C, D, E, F, G, H or I of alpha-1-microglobulin.

23. A biomarker according to any one of claims 18 to 22 wherein the isoforms or naturally occurring derivatives thereof are selected from the group comprising isoform A, B, or J of ATIII, isoform F, B or J of SAP, isoform A, C, D, E, F or G of apoJ, isoform E or G of alpha-1-microglobulin.

24. A biomarker according to any one of claims 18 to 23 wherein the isoforms or naturally occurring derivatives thereof are selected such that:
where the molecule is ATIII, the ratio is generated between at least isoforms A, B, C and J, optionally the ratio is generated between NJ, B/J or C/J; or
where the first molecule is SAP, the ratio is generated between isoforms F and J;
where the molecule is ApoJ, the ratio is generated between isoforms A, B and D; or
where the molecule is alpha-1-microglobulin, the ratio is generated between isoform E or G of alpha-1-microglobulin.

25. Use of a biomarker according to any one of claims 18 to 24 for the diagnosis, differential diagnosis and/or prognosis of a neurological disease in a subject.

26. A use according to claim 25 wherein the neurological disease is Alzheimer's disease or Parkinson's disease.

27. A method for diagnosis, differential diagnosis and/or prognosis of a neurological disease in a patient including:
(a) obtaining a first sample from the patient;
(b) isolating and identifying a molecule with heparin binding affinity from the first sample wherein the molecule is validated as a biomarker for the neurological disease according to the methods of any one of claims 1 to 17 from the sample;
(c) determining whether the patient is diagnosed, differentially diagnosed and/or prognosed with the neurological disease based on the level of the biomarker identified in step b).

28. A method for diagnosis, differential diagnosis and/or prognosis of a neurological disease in a patient including:
(a) obtaining a sample from the patient;
(b) isolating and identifying at least two related forms of a biomarker validated according to the methods of any one of claims 1 to 17 from the sample;
(c) determining a level of the biomarkers from (b);
(d) generating a ratio between the levels of the two related forms of the biomarkers identified in step (b);
(e) concluding from the ratio generated in step (d) whether the mammal is diagnosed, differentially diagnosed and/or prognosed with a neurological disease based on the ratio value compared with a reference ratio.

29. A method for diagnosis, differential diagnosis and/or prognosis of a neurological disease in a patient including:
(a) obtaining a first sample from a patient;
(b) isolating and identifying a level of a first and second biomarker with heparin binding affinity from the first sample, wherein the first and the second biomarkers are related and wherein the first and second biomarkers are validated as a biomarker for the neurological disease according to any one of claims 1 to 17,
(c) generating a ratio between the levels of the first and second biomarkers to provide a generated ratio,
(d) repeating steps (b)-(c) in a second sample obtained from a control to provide a reference ratio
(e) comparing the generated ratio identified in the first sample with the reference ratio identified in the second sample;
(f) concluding a neurological disease status based on a difference between the generated ratio and the reference ratio.

30. A method for monitoring the progression of a neurological disease in a mammal, said method comprising
(a) quantifying in a further sample obtained from a mammal previously evaluated for a neurological disease, levels of at least two related forms of a biomarker with heparin binding affinity according to any one of claims 1 to 17 that were previously evaluated in the mammal;
(b) generating a ratio between the levels of the at least two forms of the related biomarkers in step (a) to provide a generated ratio;
(c) comparing the generated ratio of step (b) with a reference ratio previously defined as characteristic for mammals diagnosed with a neurological disease; wherein the reference ratio is generated following quantifying the levels of the same related biomarkers of step (a) in a sample obtained from at least one control mammal, where at least one control mammal can be positive or negative for the neurological disease; and
(d) concluding from the comparison in step (c) whether the neurological disease status of the mammal previously evaluated for a neurological disease has changed by correlating the generated ratio of step (b) to the reference ratio in a range previously defined as characteristic for the neurological disease for the at least one control mammal.

31. A method according to claim 30 wherein the reference ratio is generated following quantifying the levels of the same related biomarkers of step (a) in a sample obtained from the mammal from an earlier time period.

32. A method for stratifying or identifying a mammal at risk of developing a neurological disease, said method comprising
(a) quantifying in a sample obtained from a mammal, levels of at least two related forms of a biomarker with heparin binding affinity according to any one of claims 1 to 17;
(b) generating a ratio between the levels of the at least two related forms of the biomarkers in step (a) to provide a generated ratio;
(c) comparing the ratio of step b) with a reference ratio previously defined as characteristic for mammals diagnosed with a neurological disease; wherein the reference ratio is generated following quantifying the levels of the same related biomarkers of step (a) in a sample obtained from at least one control mammal;
(d) concluding from the comparison in step c) whether the mammal is diagnosed, differentially diagnosed and/or prognosed with a neurological disease by correlating the generated ratio of step b) to the reference ratio in a range previously defined as characteristic for the neurological disease for the at least one control mammal; and
(e) based on the conclusion of step d) sorting the mammal into different classes of the neurological disease based on the severity of the neurological disease differentially diagnosed and/or prognosed in the mammal.

33. A method for screening for agents that interact with and/or modulate the expression or activity of a biomarker associated with a neurological disease, said method comprising:
(a) contacting a biomarker or a portion of the biomarker with heparin binding affinity as herein described with an agent;
(b) quantifying levels of at least two related forms of the biomarker;
(c) generating a ratio between the levels of the at least two related forms of the biomarkers in step (b) to provide a generated ratio;
(d) comparing the ratio of step c) with a reference ratio previously defined as characteristic for the biomarker in the absence of the agent; wherein the reference ratio is generated following quantifying the levels of the same related biomarkers of step (b) in the absence of the agent;
(e) concluding from the comparison in step d) whether or not the agent interacts with and/or modulates the expression or activity of a biomarker associated with a neurological disease by correlating the generated ratio of step c) to the reference ratio in a range previously defined as characteristic for the biomarker.

34. A method according to any one of claims 27-33 wherein the neurological disease is Alzheimer's Disease or Parkinson's Disease.

35. A method according to claim 34 wherein the biomarkers are selected from the group comprising antithrombin III, serum amyloid P, ApoJ, ANT3_HUMAN Antithrombin_III, APOH_HUMAN Beta_2_glycoprotein, FIBB_HUMAN Fibrinogen beta chain, FIBA_HUMAN Fibrinogen alpha chain, C9JC84_HUMAN Fibrinogen gamma chain, ITIH2_HUMAN Inter_alphatrypsin inhibitor heavy chain H2, HRG_HUMAN Histidine_rich glycoprotein, BOUZ83_HUMAN Complement C4 beta chain, CFAH_HUMAN Complement factor H, HEP2_HUMAN Heparin cofactor 2, and E9PBC5_HUMAN Plasma kallikrein heavy chain or their naturally occurring derivatives or isoforms thereof wherein the neurological disease is Alzheimer's Disease.

36. A method according to claim 34 wherein the biomarkers is alpha-1-microglobulin or its naturally occurring derivatives or isoforms thereof wherein the neurological disease is Parkinson's disease.

37. A method according to any one of claim 34 or 35 wherein the isoforms or naturally occurring derivatives thereof are selected from the group comprising isoforms A, B, C or J of antithrombin III, isoforms B, C, D, F, G, H or J of serum amyloid P (SAP), isoforms A, B, C, D, E, F or G of apoJ, or isoforms A, B, C, D, E, F, G, H or I of alpha-1-microglobulin.

38. A method according to any one of claims 34 to 37 wherein the isoforms or naturally occurring derivatives thereof are selected from the group comprising isoform A, B, or J of ATIII, isoform F, B or J of SAP or isoform A, C, D, E, F or G of apoJ, isoform E or G of alpha-1-microglobulin.

39. A method according to any one of claims 34 to 38 wherein the isoforms or naturally occurring derivatives thereof are selected such that:
where the molecule is ATIII, the ratio is generated between at least isoforms A, B, C and J, optionally to NJ, B/J or C/J; or
where the first molecule is SAP, the ratio is generated between isoforms F and J, or
where the molecule is ApoJ, the ratio is generated between isoforms A, B and D,
where the molecule is alpha-1-microglobulin, the ratio is generated between isoform E or G of alpha-1-microglobulin.

40. A kit for identifying a biomarker of a neurological disease including:
(a) a first component for isolating a molecule with heparin binding affinity from a sample obtained from a mammal that is positive for a neurological disease;
(b) a second component for validating the isolated molecule as a biomarker of the neurological disease wherein the second component comprises reagents to determine a level of the biomarkers that are likely to indicate that a subject possesses a neurological disease related to high amyloid loading.

41. A kit for diagnosing a neurological disease in a patient including:
(a) a first component for isolating a molecule with heparin binding affinity from a patient sample
(b) a second component for determining whether the patient is diagnosed with the neurological disease wherein the second component comprises reagents to determine a level of the biomarkers that are likely to indicate that a subject possesses a neurological disease related to high amyloid loading.

42. A kit according to claim 40 or 41 wherein the first component is a heparin sepharose column.

43. A kit according to claim 41 or 42 wherein the second component comprises reagents to quantify a level of isoforms or naturally occurring derivatives thereof selected from the group comprising isoforms A, B, C or J of antithrombin III, isoforms B, C, D, F, G, H or J of serum amyloid P (SAP), isoforms A, B, C, D, E, F or G of apoJ, or isoforms A, B, C, D, E, F, G, H or I of alpha-1-microglobulin.

44. A kit according to any one of claims 41 to 43 wherein the neurological disease is Alzheimer's Disease or Parkinson's Disease.
